(19) United States
(12) Patent Application Publication
(10) Pub. No.: US 2005/0064026 A1
(43) Pub. Date: Mar. 24, 2005

(54) Method for preparing homogenous liposomes and lipoplexes

(75) Inventors: Patrick Garidel, Norderstedt (DE); Hans Hoermann, Biberach (DE); Nicole Denkinger, Biberach (DE); Regine Peschka-Suess, Merdingen (DE); Rolf Schubert, Freiburg (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 10/925,324

(22) Filed: Aug. 24, 2004

Related U.S. Application Data
(60) Provisional application No. 60/500,735, filed on Sep. 5, 2003.

(30) Foreign Application Priority Data
Sep. 8, 2003 (EP) EP 03019663

(51) Int. Cl. A61K 9/127

(52) U.S. Cl. 424/450

(57) ABSTRACT

The present invention relates to a continuous low pressure extrusion process for preparing liposomes, processes for preparing complexes consisting of correspondingly prepared liposomes and nucleic acid molecules (lipoplexes), processes for the stable storage of corresponding lipoplexes, and correspondingly prepared liposomes and lipoplexes.

### RELATED APPLICATIONS

Benefit of U.S. Provisional Application Ser. No. 60/500,735, filed on Sep. 5, 2003, is hereby claimed, and which application is incorporated herein in its entirety.

### TECHNICAL FIELD

The present invention relates to processes for preparing liposomes and complexes consisting of liposomes and nucleic acid molecules (lipoplexes), processes for the stable storage of corresponding lipoplexes, and correspondingly prepared liposomes and lipoplexes.

### BACKGROUND TO THE INVENTION

With the advent of treatment methods using gene therapy, liposomes were recognised as being a highly promising alternative to viral gene transfer systems. The complexing of nucleic acids in/with liposomes and the use of corresponding complexes (lipoplexes) for gene therapy approaches however imposes new demands on liposome technology. For efficient and reproducible gene transfer a variety of chemical and physical parameters of the liposomes/lipoplexes have to be defined. In addition, the process must be capable of being carried out under aseptic conditions and must meet the strict manufacturing requirements for pharmaceutical compositions.

The development of cationic liposomes is a major step in the preparation of non-viral gene-therapeutically effective transfer systems. Cationic liposomes are prepared either from an individual cationic lipid or, more often, from a combination of a cationic lipid with a neutral amphiphile (helper lipid, co-lipid). The first reagent of this kind, DOTMA ([N-1-(2,3-dioleyloxy)propyl]-N,N,N,-trimethylammonium chloride), is capable of transfecting mammalian cells in vitro and in vivo (Felgner et al. (1989) Nature 337, 387-388) after being mixed with an equimolar amount of DOPE (dioleoylphosphatidylethanolamine). In the meantime, a number of cationic lipids are known which are used in gene transfer either directly or in conjunction with neutral amphiphiles. These include e.g. DORI (1,2-dioleoyloxycarbonylpropyl-3-dimethylhydroxyethylammonium bromide, DORIE (1,2-dioleyloxypropyl-3-dimethyl-hydroxyethylammonium bromide), DOTAP (dioleoyltrimethylammonium-propane-(N-[1-(2,3-dioleoyloxy)propyl]-N,N,N-trimethylammonium-methylsulphate)), DMRIE (N-(1,2-dimyristoyloxypropyl)-N,N-dimethyl-N-hydroxyethylammonium-bromide)), DOGS (di-octadecylamidologycylspermine), DOSPA (2,3-dioleyloxy-N-[2(spermine-carboxamido) ethyl]-N,N-dimethyl-1-propanaminium trifluoroacetate), PDMAEMA (poly (2-dimethyl-amino)ethyl-methacrylate), DDAB (dimethyldioctadecylammonium) DC-Chol 3β-[N-(N′,N′-dimethylaminoethyl)carbamoyl]-cholesterol) and DAC-Chol ((3-beta[N(N,N′-dimethylamino-ethane)carbamoyl]-cholesterol)). In addition there are various sperminecholesteryl-carbamates, as described for example in WO96/18372 or 1,4-dihydropyridine derivatives, as described for example in WO01/62946. An inconclusive summary of relevant cationic lipids can also be found for example in the publication by Miller (1998), Angew. Chem. 110, 1862-1880, which is specifically incorporated herein by reference. Some cationic lipids and mixtures are also commercially obtainable such as Effectene™ and SuperFect™ (Qiagen, Hilden, Germany), FuGene 6™ (Roche, Mannheim, Germany), LipoFectin™, LipoFectin2000™, LipoFECTAMINE PIuS™ (Invitrogen, Karlsruhe, Germany).

Since the end of the 80s lipofection, i.e. the transfection of nucleic acid complexed in or with liposomes has been promoted and successfully tried out on many cell types and cell lines. For human use in gene therapy it has been found that the use of lipoplexes is a highly promising method. (Galanis (2002) Current Opinion Molec. Therapeutics 4: 80-87; Stopeck et al. (2001) Clinical Cancer Res. 7: 2285-2291.; Voges et al. 2002) Human Gene Therapy. 13: 675-685; Jacobs et al. (2001) Lancet. 358: 727-729; Morgtan (ed.) Gene Therapy Protocols (2002) Humana Press Inc. New Jersey).

For human use a simple and practical formulation is needed. The requirement is particularly for the manufacture of physically and chemically stable lipoplexes and the establishing of a process for reproducibly producing these complexes with constant quality in terms of their biophysical and biological properties.

The combining of cationically charged amphiphiles such as e.g. lipids and negatively charged nucleic acid molecules leads to the formation of complexes via electrostatic interactions. Depending on the mixing process, the concentration of the starting materials, the formulation, etc., the complex may be flocculated (Gershon et al. (1993) Biochemistry 32: 7143-7151; Lasic et al. (1997) J. Am. Chem. Soc. 119: 832-833; Eastman et al. (1997) BBA 1325: 41-62). The dispersion is thus not stable and the product is unsuitable for clinical applications as the aggregates formed are very large (ranging from several microns to millimetres). As it is very difficult to stabilise lipoplexes with regard to their aggregation characteristics, for clinical use the lipoplexes are freshly prepared by the doctor in the hospital (“bed-side”) or stored in frozen form (Galanis (2002) supra; Stopeck et al. (2001) supra; Voges et al. (2002) supra; Jacobs et al. (2001) supra; Morgtan (2002) supra, Gao & Huang (1995) Gene Therapy 2: 710-722; Feigner et al. (1995) supra; Nabel et al. (1994) Hum. Gene Therapy 5: 57-77 and 1089-1094). For this reason it is very important to develop processes which provide a reproducible quality of lipoplex.

The biophysical properties of the lipoplexes depend among other things on the properties and quality of the liposomes which are used for complexing nucleic acid. Various methods of preparing liposomal suspensions and liposome preparations are described in the literature. Thus for example liposomes may be prepared by sonicating lipid-containing solutions by means of an ultrasound bath or an oscillating rod. The methods involved are very energy-intensive methods (Perrett et al., (1991) J. Pharmacy & Pharmacology, 43: 154-161), which cannot easily be scaled up for pharmaceutical production. There is also the danger that by using the oscillating rod the liposomes will be contaminated by small particles of metal. The product quality (size of the liposomes) is difficult to reproduce and the liposomes thus formed are usually very small (≦250 nm). Gregoriadis et al. (1990) Int. J. Pharmaceutics 65: 235-242 describe how liposomes may be formed using a dehydration-hydration method. Active substances may also be included in the liposomes. The lipid suspensions formed do however exhibit a high degree of inhomogeneity regarding their size distribution and polydispersity. More homogeneous lipid suspensions are only obtained after an additional process step, microfluidisation (Washington & Davis, (1988) Int. J. Pharmaceutics 44: 169-176; Vuillemard J C. (1991) J. Microencapsulation 8: 547-562). However, this method operates at very high pressures.

The preparation of liposomes by extrusion processes is known for the preparation of phospholipid dispersions (Elorza et al. (1993) J.

Microencapsulation 10: 237-248; Berger et al. (2001) Int. J. Pharmaceutics 223: 55-68). The work is generally done under very high pressures, particularly if the extrusion is done through membranes with a pore size of less than 1000 nm (in the case of Berger et al. (supra) at operating pressures in excess of 50×10⁵ Pa). This leads on the one hand to small liposomes and on the other hand involves considerable expenditure to convert it into a large-scale process. This extrusion process is often also combined with another process step, e.g. a freeze-thaw step, in order to increase the product quality (Mayer et al. 1986) BBA 858: 161-168; Hope et al. (1985) BBA 812: 55-65; Nayar et al. (1989) BBA 986: 200-206) describe a method of extruding gel phase lipids but this also requires very high pressures of between 17.5 and 49×10⁵ Pa. Depending on the lipid used and the extrusion pressure the authors mainly describe the preparation of liposomes with very small diameters, generally less than 200-150 nm.

Sorgi & Huang (1996) Int. J. Pharmaceutics 144: 131-139, who describe the preparation of cationic liposomes using the microfluidiser, also operate at high pressures. The operating pressure used was roughly 6.2×10⁵ Pa. The diameter of the liposomes produced was less than 200 nm (cf. also WO96/27393). Patent Application WO98/17814 also describes cationic liposomes roughly 800 nm in size.

To sum up it can be stated that the methods known in the art either lead to very small liposomes (<200 nm), which can only be used in very restricted circumstances for the transfer of nucleic acids owing to their low transfection efficiency, or to inhomogeneous liposomes which are indeed sufficiently transformable but are not stable over long periods and may not meet the strict quality requirements for pharmaceutical compositions.

Consequently, one aim of the present invention was to provide a process for preparing homogeneous liposomes which are highly stable on storage, and enable homogeneous lipoplexes to be produced which have sufficient transfection efficiency and at the same time good stability. A further aim was to provide corresponding liposomes measuring 250-800 nm and lipoplexes measuring 250-600 nm in size.

A further aim of the present invention was to discover a corresponding process for preparing liposomes or lipoplexes, in which liposomes or lipoplexes can be produced under GMP conditions. This refers to the production of reproducibly homogeneous liposome/lipoplex batches under aseptic conditions on a larger scale.

A further aim of the present invention was to prepare corresponding homogeneous and storable liposomes and lipoplexes which have sufficient transfection efficiency and at the same time good stability and GMP quality.

### SUMMARY OF THE INVENTION

The present invention relates to a process for preparing homogeneous liposomes, wherein a lipid suspension is extruded through a porous membrane, preferably with a pore size of between 600-900 nm, in a continuous process, under low pressure conditions at less than 3×10⁵ Pa. It has been found that a corresponding process using cationic liposomes, or liposomes containing a cationic lipid and a neutral amphiphile (e.g. consisting of DC-Chol/DOPE or DAC-Chol/DOPE) leads to stable and homogeneous liposome mixtures with liposomes measuring 250-800 nm, preferably 250-600 nm and a polydispersity index of ≦0.6, preferably ≦0.5, more preferably ≦0.4. It has proved particularly advantageous to use a polycarbonate membrane as the extrusion membrane.

According to a preferred embodiment the concentration of the liposomes in the lipid suspension is between 0.04 and 5 mg/ml, preferably between 0.1-2 mg/ml, particularly between 0.1-1 mg/ml. In this context, flow rates of between 10-250 ml/min, preferably between 50-150 ml/min, most preferably between 75-120 ml/min have proved particularly suitable. The extrusion process according to the invention can also be carried out at ambient temperature without affecting the quality of the liposomes.

According to another embodiment the corresponding process according to the invention is carried out in a sealed system under aseptic conditions. The liposomes or the liposome suspension can be pre-filtered beforehand through membranes with a pore size up to 1000 nm.

According to another embodiment the present invention relates to a process for the continuous low pressure extrusion of liposomes, preferably liposomes which contain a cationic lipid or a mixture of a cationic lipid and a neutral amphiphil, preferably a combination of DC-Chol or DAC-Chol and DOPE, at a pressure below 3×10⁵ Pa, a flow rate between 10-250 ml/min and a lipid concentration between 0.04-5 mg/ml, characterised in that the lipid suspension is continuously extruded between 2-20 times through the porous membrane. This process surprisingly produced particularly homogeneous liposomes measuring 250-600 nm, preferably 280-500 nm, most preferably 280-400 nm, with a polydispersity index of ≦0.5, preferably ≦0.4.

It has proved particularly suitable to use an apparatus as shown in FIG. 1 for the large-scale manufacture of corresponding liposomes according to the invention. The apparatus consists of a mechanical or electric pump (1), a throughflow measuring regulator (2), a manometer for measuring the filtration pressure (3), a filter holder with an extrusion membrane (pore size e.g. 600 nm/Ø e.g. 47 mm) (4) and ventilating valve (8), a temperature measuring device (5), a holding vessel (6) and an annular tubing system (7) which allows the substance to flow continuously through the extrusion membrane and to be refluxed into the holding vessel.

The present invention also relates to liposomes or liposome mixtures containing liposomes which are prepared by one of the processes according to the invention, described here. The present invention particularly relates to liposome mixtures consisting of liposomes with a defined size of between 250 and 800 nm, preferably between 250 and 600 nm, wherein the liposomes contain a cationic lipid and a neutral amphiphile and are characterised in that the polydispersity index of the liposome mixture has a value of ≦0.60, preferably ≦0.50, most preferably ≦0.4. According to another embodiment the liposome mixture is characterised in that the cationic lipid is a cholesterol such as for example DC-Chol ((3-beta[N(N′,N′-dimethylaminoethane)carbamoyl]-cholesterol)) or DAC-Chol ((3-beta[N(N,N′-dimethylamino-ethane)carbamoyl]-cholesterol)). According to another preferred embodiment the liposomes according to the invention contain an ethanolamine derivative, for example dioleoylphosphatidylethanolamine (DOPE)).

Furthermore, the present invention relates to a process for mixing liposomes according to the invention with nucleic acid molecules (nucleic acid molecule=nucleic acids). It has proved particularly advantageous to carry out the mixing through a so-called Y-shaped member, which enables the liposomes and nucleic acid molecules to be combined evenly and continuously. Moreover, it has been found that a liposome-nucleic acid charging ratio of between +/−4-0.01, preferably between +/−1.25-0.75, produces particularly stable and homogeneous lipoplexes. It has proved particularly advantageous to combine equal volumes of a suspension containing liposomes and the solution containing nucleic acid.

According to another embodiment the concentration of the liposomes when mixing the liposomes and nucleic acid is between 0.02-1 mg/ml. Flow rates of between 100-500 ml/min have proved advantageous when mixing the liposomes and nucleic acids. The corresponding process can be carried out in a sealed apparatus, so that the lipoplexes can be produced under aseptic conditions.

The corresponding process according to the invention makes it possible to prepare a lipoplex mixture consisting of lipoplexes with a defined size of between 250-600 nm, preferably between 275-500 nm, most preferably between 275-400 nm, and with a polydispersity index of ≦0.50, preferably ≦0.40. According to another embodiment, therefore, the present invention relates to lipoplexes which are prepared by the process according to the invention described here, particularly those with a defined size of between 250-600 nm, preferably between 275-500 nm, most preferably between 275-400 nm, and with a polydispersity index of ≦0.50, preferably ≦0.40.

In another embodiment the present invention relates to a process for long-term storage of correspondingly prepared lipoplexes, for example by lyophilisation in the presence of a suitable stabiliser, comprising the steps of (a) freezing the lipoplex mixture to a temperature of ≦−50° C.; (b) drying the lipoplex mixture at approximately −20° C. for at least 35 hours, (c) drying the lipoplex mixture at approximately 20° C. for at least 10 hours.

According to a preferred embodiment the process for lyophilising the lipoplex mixture according to the invention in the presence of a suitable stabiliser comprises the following steps: (a) freezing the lipoplex mixture to a temperature of ≦−50° C. at a temperature lowering rate of approximately ≦1° C./min; (b) incubating the lipoplex mixture at ≦−50° C. for at least 2 hours; (c) heating the lipoplex mixture to approximately −20° C. at a heating rate of approximately ≦0.3° C./min; (d) drying the lipoplex mixture at approximately −20° C. for at least 35 hours; (e) heating the lipoplex mixture from about −20° C. to about 20° C. at a heating rate of approximately ≦0.44° C./min; and (f) drying the lipoplex mixture at about 20° C. for at least 10 hours. It has proved particularly suitable to carry out the drying in point (d) at a pressure of between 0.01-0.1 mbar, preferably between 0.025-0.05 mbar.

Moreover the present invention also relates to lipoplex lyophilisates which may be obtained by one of the processes according to the invention described here, as well as the use of the homogeneous lipoplexes or lipoplex lyophilisates described here for the preparation of pharmaceutical compositions or as pharmaceutical compositions in gene therapy for the transfection of mammalian cells.

### DESCRIPTION OF THE FIGURES

FIG. 1: Diagrammatic structure of a continuous extrusion apparatus consisting of a hose pump (1), flow regulator (2), manometer for measuring the filtration pressure (3), filter holder with extrusion membrane of a specified pore size, e.g. 600 nm Ø 47 mm (4) and ventilating valve (8), temperature measuring device (5), a holding vessel (6) and an annular tubing system (7).

FIG. 2: Flow diagram for preparing lipoplexes of a defined particle size.

FIG. 3: Diagram of the process for preparing storable lipoplexes. Holding vessel for lipid suspension (1), a first pump (2), extrusion apparatus with a porous membrane of 600-900 nm (3), valves (4), branch (5), Y-shaped member (6), holding vessel for nucleic acids (7), a second pump (8) and a collecting container for lipoplexes (9).

FIG. 4: Flow diagram for the preparation of DC30/nucleic acid lipoplexes in a ratio of dimensions of 4:1. Batch size: 3750 ml bulk, dose: 0.025 mg/ml nucleic acid.

FIG. 5: Flow diagram for the preparation of DAC30/nucleic acid lipoplexes in a ratio of dimensions of 5:1. Batch size: 700 ml bulk, dose: 0.025 mg/ml nucleic acid.

FIG. 6: (A) SEM (Scanning Electron Microscope) photograph of the surface of the lyophilisation cake. (B) SEM (Scanning Electron Microscopy) photograph within the lyophilisation cake.

FIG. 7: Effect of the different mixing sequence during the lipoplex preparation of lipofectin and pAH7-EGFP plasmid in a mass ratio of 6:1 (w/w) on the transfection efficiency and size of the lipoplex (aggregate). LtoD=lipid to DNA, DtoL=DNA to lipid. The lipoplex size was determined by PCS. The transfection efficiency was tested on A-10 SMC (smooth muscle cells).

FIG. 8: Bioactivity (transfection efficiency) of DAC30/pMCP-1 5:1 (w/w) lipoplexes stored at 4° C. for several months. The transfection efficiency is based on an internal standard.

FIG. 9: Y-shaped member for mixing nucleic acid and liposomes.

### DETAILED DESCRIPTION OF THE INVENTION

The present invention relates to a process for preparing homogeneous liposomes, wherein a lipid suspension is extruded through a porous membrane with a pore size of preferably 600-900 nm in a continuous process, characterised in that the extrusion is carried out under low pressure conditions at pressures below 3×10⁵ Pa. The correspondingly prepared liposomes have an average size of 250-800 nm. The polydispersity index of the liposome mixture is ≦0.6, preferably ≦0.5, or ≦0.4.

By the term “liposomes” is meant an aqueous lipid-containing suspension of multi-layered (consisting of at least a double layer of lipid) generally spherical accumulations of lipid molecules which are formed by mechanically mixing a dry lipid in water.

The “polydispersity index” (=PI) is a measurement of the homogeneous or heterogeneous size distribution of the individual liposomes in a liposome mixture and indicates the breadth of the particle distribution in a mixture. A precise definition can be found in the chapter “Material and methods”. The PI can be determined, for example, by the method mentioned in the chapter “Material and methods” which serves as a reference method here.

The term “low pressure conditions” in the sense of the invention, refers to filtration pressures of less than 3×10⁵ Pa, preferably less than 2×10⁵ Pa and particularly preferably less than 1×10⁵ Pa.

The extrusion through a membrane with a defined pore size makes it possible to prepare liposomes of a defined size. It has been found that not only the pore size but also further parameters, such as pressure, flow rate, lipid concentration affect the chemical-physical properties of the extruded liposomes. The extrusion at high pressures (above 3×10⁵ Pa) leads to liposomes with relatively small diameters of approximately less than 200 nm. Corresponding liposomes exhibit very low transfection efficiency after being mixed with nucleic acids, and this greatly restricts their suitability in the field of gene therapy. Moreover, a process which requires very high operating pressures can only be scaled up to an industrial scale at considerable technical expense.

With the present invention we have succeeded in providing a process for the preparation of homogeneous liposomes with a defined size of between 250-800 nm, preferably between 280-700 nm, most preferably between 280-600 nm. The process according to the invention is characterised by a high degree of reproducibility, which in turn allows the preparation of homogeneous batches of liposomes for the purposes of gene therapy. By a batch is meant liposomes or liposome mixtures which are prepared from a defined amount of starting material during an operation/production run. It has been found that the high quality (=homogeneity and stability) of the liposomes according to the invention is positively influenced by the selected process parameters and the guidance of the process.

Surprisingly it has been found that the extrusion of a lipid suspension with a lipid concentration of 0.04-5 mg/ml, preferably of 0.1-2 mg/ml, most preferably of 0.1-1 mg/ml, still more preferably 0.25-1 mg/ml leads to particularly homogeneous liposomes/liposome mixtures if it is carried out under low pressure conditions through a membrane with a pore size of 600-900 nm. Consequently, the present invention also relates to a process for preparing homogeneous liposomes, wherein a lipid suspension is extruded through a porous membrane with a pore size of 600-900 nm in a continuous process under low pressure conditions at less than 3×10⁵ Pa, characterised in that the lipid concentration is 0.04-5 mg/ml, preferably 0.1-2 mg/ml, most preferably 0.1-1 mg/ml, still more preferably 0.25-1 mg/ml.

It has become apparent that in addition to the pore size, the filtration pressure and the lipid concentration the flow rate also affects the homogeneity of the liposomes/liposome mixture. Surprisingly flow rates of between 10-250 ml/min, preferably between 50-150 ml/min, most preferably between 75-120 ml/min produce particularly homogeneous liposomes. Consequently the present invention also relates to processes for preparing homogeneous liposomes by low pressure extrusion through a 600-900 nm membrane, characterised in that the lipid concentration is between 0.04-5 mg/ml, preferably between 0.1-2 mg/ml, most preferably between 0.1-1 mg/ml, still more preferably between 0.25-1 mg/ml and the flow rate during the extrusion is between 10-250 ml/min, preferably between 50-150 ml/min, most preferably between 75-120 ml/min.

Preferably the extrusion membrane is a polycarbonate membrane with a pore size of 600-900 nm, for example with a pore size of 600, 650, 750, 800, 850, or 900 nm. However, extrusion membranes made from other materials, e.g. from polymers with suitable properties, are also suitable for the purposes of the invention.

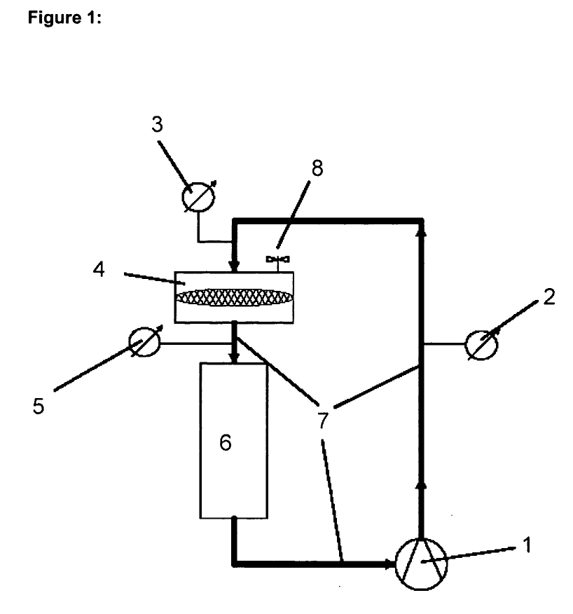

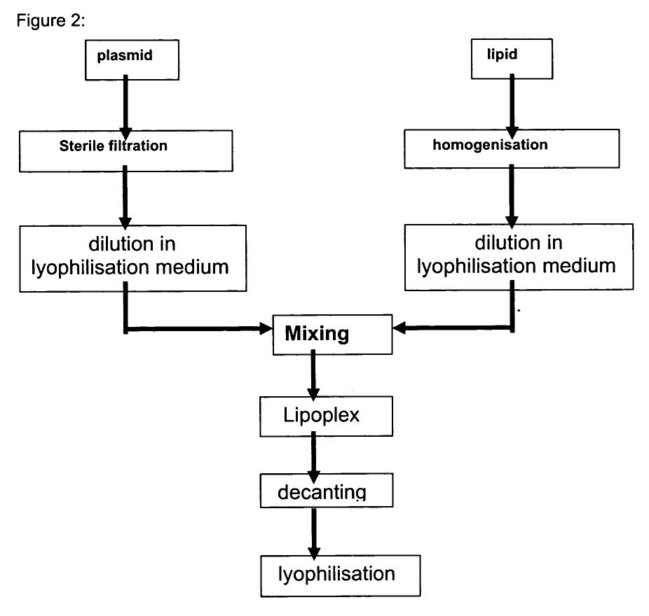

Moreover it has been found that the quality of the liposomes, particularly the homogeneity of the liposome mixture, could be improved still further if the extrusion is carried out in a continuous process and the lipid suspension is extruded several times through the extrusion membrane, preferably between 2 -20 times, (cf. e.g. Embodiment 1, Table 4). Consequently the present invention also relates to processes for preparing homogeneous liposomes by low pressure extrusion of a lipid suspension, preferably with a lipid concentration between 0.04-5 mg/ml, preferably between 0.1-2 mg/ml, most preferably between 0.1-1 mg/ml, still more preferably between 0.25-1 mg/ml, through a 600-900 nm membrane, preferably with a flow rate between 10-250 ml/min, most preferably between 50-150 ml/min, more preferably between 75-120 ml/min, characterised in that the lipid suspension is extruded through the membrane at least twice, preferably between 2 and 20 times. Particularly preferred is a corresponding process wherein the extrusion is carried out in a continuous process. Even more preferred is a corresponding extrusion method which is carried out in a sealed system, as shown in FIG. 1, for example, under aseptic conditions.

Surprisingly it has been found that the process according to the invention is particularly suitable for preparing homogeneous cationic liposomes/liposome mixtures or liposomes/liposome mixtures containing a cationic lipid and a neutral amphiphil. In particular, liposomes consisting of a mixture of neutral amphiphile and cationic lipid have proved particularly homogeneous and stable, if they were prepared by the process according to the invention described here.

By a “cationic lipid” is meant a lipid which has a positive excess charge under specified conditions. By a neutral (zwitterionic) amphiphile is meant a molecule which has no excess charge under specified conditions and is hence charge-neutral.

Consequently according to another embodiment the present invention also relates to processes for preparing homogeneous liposomes/liposome mixtures containing at least one cationic lipid or a cationic lipid and a neutral amphiphil. Suitable cationic lipids for the purposes of the invention are for example DOTMA [N-1-(2,3-dioleyloxy)propyl]-N,N,N,-trimethylammonium chloride), DORI (1,2 -dioleoyloxycarbonylpropyl-3-dimethylhydroxyethylammonium bromide, DORIE (1,2-dioleyloxypropyl-3-dimethylhydroxyethylammonium bromide), DOTAP (dioleoyltrimethylammoniumpropane(N-[1-(2,3-dioleoyloxy)propyl]-N,N,N-trimethylammonium-methylsulphate)), DMRIE (N-(1,2-dimyristoyloxypropyl)-N,N-dimethyl-N-hydroxyethylammonium-bromide)), DOGS (dioctadecylamidologycyl spermine), DOSPA (2,3-dioleyloxy-N-[2(sperminecarboxamido)ethyl]-N,N-dimethyl-1-propanaminium trifluoroacetate), PDMAEMA (poly(2-dimethyl-amino)ethyl methacrylate), DDAB (dimethyldioctadecylammonium) DC-Chol 3β-[N—(N′,N′-dimethyl aminoethyl)carbamoyl]-cholesterol) and DAC-Chol ((3-beta[N(N,N′-dimethylamino-ethane)carbamoyl]cholesterol)). In addition there are various sperminecholesteryl-carbamates, as described for example in WO96/18372, or 1,4-dihydropyridine derivatives, as described for example in WO01/62946. An inexhaustive overview of relevant cationic lipids can also be found for example in the publication by Miller (1998), Angew. Chem. 110, 1862-1880, which is specifically incorporated herein by reference. Preferably the process according to the invention is suitable for preparing homogeneous cholesterol-containing liposomes/liposome mixtures, preferably liposomes/liposome mixtures which contain DAC-Chol or DC-Chol as cationic lipid. A description of DAC-Chol can also be found inter alia in WO96/20208, Reszka et al., while a description of DC-Chol can be found in U.S. Pat. No. 5,283,185, Epand et al. Some cationic lipids and mixtures are also commercially obtainable such as Effectene™ and SuperFect™ (Qiagen, Hilden, Germany), FuGene 6™ (Roche, Mannheim, Germany), LipoFectin™, LipoFectin2000™, LipoFECTAMINE Plus™ (Invitrogen, Karlsruhe, Germany).

Suitable neutral amphiphiles for the purposes of the invention are for example choline derivatives such as dimyristoylphosphatidylcholine (DMPC), dipalmitoyl-phosphatidylcholine (DPPC), dioleoylphosphatidylcholine (DOPC) o r ethanolamine derivatives such as dimyristoylphosphatidyl ethanolamine (DMPE), dipalmitoylphosphatidyl ethanolamine (DPPE), dioleoylphosphatidylethanolamine (DOPE), of which DOPE is particularly preferred.

Surprisingly it has been found that the process according to the invention described here leads to particularly homogeneous liposomes/liposome mixtures with low polydispersity if the lipid suspension to be extruded contains DOPE as a neutral amphiphile and DC-Chol, preferably DAC-Chol, as a cationic lipid.

Cationic lipid and neutral amphiphile may be present in a ratio by weight of 1:99 to 99:1, which is intended to include all the ratios by weight between these values. Particularly stabile and homogeneous liposomes are obtained if the cationic lipid and the neutral amphiphile are mixed in a ratio by weight of 10:90 to 40:60, for example 10:90, 15:85, 20:80, 25:75, 30:70, 35:65, 40:60. Also preferred are mixing ratios of 21:79, 22:78, 23:77, 24:76, 25:75, 26:74, 27:73, 28:72, 29:71, 30:70, 31:69, 32:68, 33:67, 34:66, 35:65, 36:64, 37:63, 38:62, 39:61, 40:60, particularly a mixing ratio of 30:70 in the case of lipid mixtures of DC-Chol:DOPE or DAC-Chol:DOPE. DC-Chol:DOPE or DAC-Chol:DOPE in a weight ratio of 70:30 in each case are hereinafter also referred to as DC30 in the case of DC-Chol:DOPE as and DAC30 in the case of DAC-Chol:DOPE. Accordingly in a particularly preferred embodiment the present invention relates to the preparation of homogeneous liposomes with a low polydispersity index of preferably ≦0.6, more preferably ≦0.5, and still more preferably ≦0.4, consisting of DC-Chol:DOPE, preferably DAC-Chol:DOPE in a ratio by weight of 30:70 (DC30 or DAC30).

According to another embodiment of the process according to the invention the lipid suspension to be extruded is a suspension of a cationic lipid and a neutral amphiphile in an aqueous solution. The lipid suspension can additionally contain other substances, for example salts, polymers, sugars or sugar alcohols. The addition of corresponding substances (=adjuvants) can further improve the stability of the liposomes which are to be prepared as well as the liposome-nucleic acid complexes prepared from them.

Examples of polymers include polyvinylpyrrolidones, derivatised celluloses such as e.g. hydroxymethyl, hydroxyethyl, or hydroxypropylethyl cellulose, polymeric sugars such as e.g. ficoll or dextran, starch such as e.g. hydroxyethyl or hydroxypropyl starch, dextrins such as e.g. cyclodextrin (2-hydroxypropyl-β-cyclodextrin, sulphobutylether-β-cyclodextrin), polyethylenes, glycols, chitosan, collagen, hyaluronic acid, polyacrylates, polyvinylalcohols and/or pectins. Sugar may for example be mono-, di-, oligo- or polysaccharides or a combination thereof. Examples of monosaccharides are fructose, maltose, galactose, glucose, D-mannose, sorbose and the like. Disaccharides are for example lactose, sucrose, trehalose, cellobiose and the like. Examples of suitable polysaccharides include in particular raffinose, melecitose, dextrin, starch and the like. Examples of sugar alcohols include, in addition to mannitol, xylitol, maltitol, galactitol, arabinitol, adonitol, lactitol, sorbitol (glucitol), pyranosylsorbitol, inositol, myoinositol and the like.

Examples of salts include in particular pharmaceutically acceptable salts, such as for example inorganic salts such as chlorides, sulphates, phosphates, di-phosphates, hydrobromides and/or nitrate salts. The lipid suspension may also contain organic salts, such as e.g. malate, maleate, fumarate, tartrate, succinate, ethylsuccinate, citrate, acetate, lactate, methanesulphonate, benzoate, ascorbate, paratoluenesulphonate, palmoate, salicylate, stearate, estolate, gluceptate or labionate salts.

The preparation method according to the invention can be used to prepare homogeneous liposome mixtures consisting of liposomes with a defined size of between 250-800 nm, preferably between 280-600 nm, most preferably between 280-500 nm, more preferably between 280-400 nm, while the liposomes preferably contain a cationic lipid and a neutral amphiphil, characterised in that the polydispersity index of the liposome mixture has a value of ≦0.60, preferably ≦0.50, more preferably ≦0.40. Consequently the present invention also relates to corresponding liposome mixtures consisting of liposomes with a defined size of between 250 and 800 nm, preferably between 280-600 nm, most preferably between 280-500 nm, more preferably between 280-400 nm, the liposomes containing a cationic lipid and a neutral amphiphil, characterised in that the polydispersity index of the liposome mixture has a value of ≦0.60, preferably ≦0.50, more preferably ≦0.40. Preferably the liposomes according to the invention contain a cholesterol derivative such as e.g. DC-Chol or DAC-Chol in combination with a neutral amphiphile selected from DMPC, DPPC, DOPC, DMPE, DPPE, or preferably in combination with DOPE. It has proved particularly advantageous to use corresponding liposomes/liposome mixtures which contain or consist of DOPE as neutral amphiphile and DC-Chol and/or DAC-Chol as cationic lipid, while the mass ratio of DOPE to the cationic lipid is 70:30 (DC30 or DAC30). Moreover, the process according to the invention may be used to prepare liposomes/liposome mixtures which contain or consist of the above-mentioned cationic lipids, neutral amphiphiles, salts, polymers, sugars, sugar alcohols or a combination thereof.

The liposomes according to the invention described here are suitable for preparing homogeneous liposome-nucleic acid complexes, to form so-called lipoplexes, by simply mixing the corresponding liposomes with nucleic acid molecules. The nucleic acid molecules (=nucleic acids) are usually genomic DNA, cDNA, synthetic DNA, RNA, mRNA, ribozyme, antisense-RNA, synthetic peptide nucleotides and single-stranded oligonucleotides, preferably cDNA. The nucleic acid may for example be contained in a DNA expression vector or in an expression cassette and in this way allow recombinant expression of a gene of interest after transfection in a target cell. In this way various genes, preferably therapeutic genes, may be locked into a target cell and expressed therein. Examples of therapeutic genes include for example insulin, insulin-like growth factor, human growth hormone (hGH) and other growth factors, tissue plasminogen activator (tPA), erythropoietin (EPO), cytokines, for example interleukins (IL) such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN)-alpha, -beta, -gamma, -omega or -tau, tumour necrosis factor (TNF) such as e.g. TNF-alpha, -beta or -gamma, TRAIL, G-CSF, GM-CSF, M-CSF, MCP-1 to MCP-5, eNOS, iNOS, HO-1, HO-2, HO-3 and VEGF, HGF.

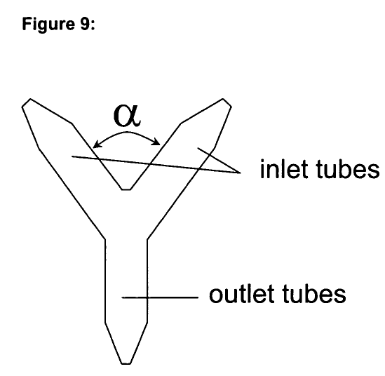

The lipoplexes are normally prepared by the addition of nucleic acid molecules to liposomes or, conversely, by the addition of liposomes to nucleic acid molecules. Within the scope of the present invention it has proved particularly advantageous to combine the liposomes and nucleic acids evenly, for example using a so-called Y-shaped member. By a Y-shaped member is meant a three-legged tube as shown in FIG. 9 for example. It consists of two inlet tubes which converge at an acute angle into an outlet tube. The continuous mixing process produces lipoplexes with a constant content of nucleic acid. Moreover, it has surprisingly been found that the continuous combining of nucleic acids and liposomes leads to almost total internalisation of the nucleic acids in the liposomes, while when nucleic acids and liposomes are simply mixed together the nucleic acids generally protrude from the liposomes and are thus less well protected and hence have a tendency to interact and induce the flocculation of the lipoplexes. Consequently the transfection of stable lipoplexes produced by continuous mixing through a so-called Y-shaped member makes it possible to improve expression in the transfected target cells (=improved bioactivity). Correspondingly prepared lipoplexes are characterised by high homogeneity, physical stability in solution, very good long-term stability and lyophilisability and at the same time a high bioactivity. In addition, the continuous combining of nucleic acid and liposomes makes it possible to prepare large amounts of lipoplexes, with a constant high quality, up to the litre scale.

Consequently, in another embodiment, the present invention relates to a process for preparing lipoplexes, characterised in that the mixing of liposomes and nucleic acid molecules is done through a Y-shaped member, which enables the liposomes and nucleic acid molecules to be combined evenly and continuously. By means of the corresponding process, preferably by using a Y-shaped member, in which the two inlet tubes through which the liposome suspension and the nucleic acids are combined are at an acute angle of <80, preferably <70, more preferably <60, still more preferably <50 or <40 degrees to one another (cf. FIG. 9, angle α), homogeneous lipoplexes measuring 250-600 nm with a polydispersity of ≦0.5, preferably ≦0.4 can be obtained. The internal diameter of the tube is dependent in each case on the volumes of liposomes and nucleic acid which are passed through the Y-shaped member together. Within the scope of the present invention Y-shaped members with an internal tube diameter of 3 to 5 mm have proved advantageous. At flow rates of 20-800 ml/min, preferably at 100-500 ml/min, corresponding Y-shaped members allow the continuous complexing of liposomes and nucleic acid to produce homogeneous lipoplexes of corresponding size. The lipoplexes shown in the Examples were mixed, for example, at a flow rate of approximately 150-170 ml/min or approximately 400 ml/min.

Within the scope of the present invention, cationic liposomes, or liposome mixtures consisting of a neutral amphiphile and a cationic lipid, such as for example DC30 or DAC30, in particular, were used to prepare the lipoplexes. The corresponding liposomes thus carry positive charges on their surface, whereas the nucleic acids are negatively charged by virtue of their phosphate skeleton. It was known from WO98/01030 that a charge ratio of positively-charged liposomes to negatively-charged nucleic acid of 1:20 (+/−), preferably 2:10 (+/−) has a stabilising influence on the lipoplexes formed. In the present case it has been found that a liposome-nucleic acid charge ratio (+/−) of 4-0.01, for example 4, 3.9, 3.8, 3.7, 3.6, 3.5 etc., 3.0, 2.9, 2.8, 2.7, 2.6, 2.5 etc., 1.0, 1.9, 1.8, 1.7, 1.6, 1.5 etc., 0.9, 0.8, 0.7, 0.6, 0.5 etc, 0.09, 0.08, 0.07, 0.06 etc. is particularly advantageous and improves the stability of the resulting lipoplexes. The liposome-nucleic acid charge ratio (+/−) indicates the ratio of positive charge of the cationic lipid used to the negative charges of the nucleic acid. It is assumed that all monovalent cationic lipids have one (1) positive charge. This means that the number of moles of cationic lipid put in corresponds to the number of moles of positive charges (this applies to a lipid which has only one (1) positive charge; for polyvalent cationic lipids this has to be taken into consideration in the calculation). The charge carriers of the negative charge on the nucleic acid are the phosphate groups (one negative charge per phosphate group). A liposome-nucleic acid charge ratio (+/−) of 1.25-0.75 has proved particularly advantageous, especially in connection with the use of DC30 liposomes or preferably with the use of DAC30 liposomes. Consequently the present invention also relates to a process for preparing homogeneous lipoplexes from cationic liposomes, or from liposomes which contain a cationic lipid and a neutral amphiphil, such as for example DC30 or DAC30, using a Y-shaped member, characterised in that the liposome-nucleic acid charge ratio (+/−) is 4-0.01, preferably 2-0.1, most preferably 1.5-0.5 and still more preferably 1.25-0.75.

Apart from the mixing process itself, the flow rate and the liposome-nucleic acid charge ratio (+/−), the stability of the lipoplexes during the mixing process can be positively affected by the liposome concentration used. It has been found that, when cationic liposomes, e.g. DC30 or preferably DAC30, are continuously mixed with nucleic acids through a Y-shaped member at a continuous flow rate of 20-800 ml/min, preferably 100-500 ml/min, and at a liposome-nucleic acid charge ratio (+/−) of 4-0.01, preferably 2-0.1, most preferably 1.5-0.5 and still more preferably 1.25-0.75, it is particularly advantageous to use a homogeneous liposome suspension with a liposome concentration of between 0.02 and 1 mg/ml. Consequently, in another aspect, the present invention relates to a process for preparing lipoplex mixtures by continuously mixing cationic liposomes, e.g. DC30 or preferably DAC30, with nucleic acids through a Y-shaped member at a continuous flow rate of 20-800 ml/min, preferably 100-500 ml/min and at a liposome-nucleic acid charge ratio (+/−) of 4-0.01, preferably 2-0.1, most preferably 1.5-0.5 and still more preferably 1.25-0.75, characterised in that a homogeneous liposome suspension with a liposome concentration of approximately 0.02-1 mg/ml, preferably approximately 0.1-0.5 mg/ml is used.

According to another preferred embodiment the processes for preparing the liposomes and the mixing of the liposomes and nucleic acids can be directly coupled to each other. Therefore, according to a particularly preferred embodiment; the present invention relates to a process for preparing homogeneous lipoplex mixtures with lipoplexes measuring 250-600 nm, preferably 275-500 nm, most preferably 275-400 nm and preferably a polydispersity index of ≦0.5, most preferably ≦0.4, comprising the steps of: (a) extruding a lipid suspension containing a cationic lipid or a mixture of a cationic lipid and a neutral amphiphil, for example DC30 or preferably DAC30, in a continuous process through a 600-900 nm membrane, preferably at a flow rate between 10-250 ml/min, most preferably between 50-150 ml/min, more preferably between 75-120 ml/min, while the lipid concentration in the lipid suspension is preferably between 0.04-5 mg/ml, preferably between 0.1-2 mg/ml, most preferably between 0.1-1 mg/ml, still more preferably between 0.25-1 mg/ml, and the lipid suspension is extruded through the membrane at least once, but preferably continuously between 2 and 20 times; a n d (b) mixing the liposome mixture thus prepared with nucleic acid molecules which have previously been filtered sterile, preferably through a 0.2 μm filter, using a Y-shaped member at a continuous flow rate of 20-800 ml/min, preferably 100-500 ml/min and at a liposome-nucleic acid charge ratio (+/−) of 4-0.01, preferably 2-0.1, most preferably 1.5-0.5 and still more preferably 1.25-0.75.

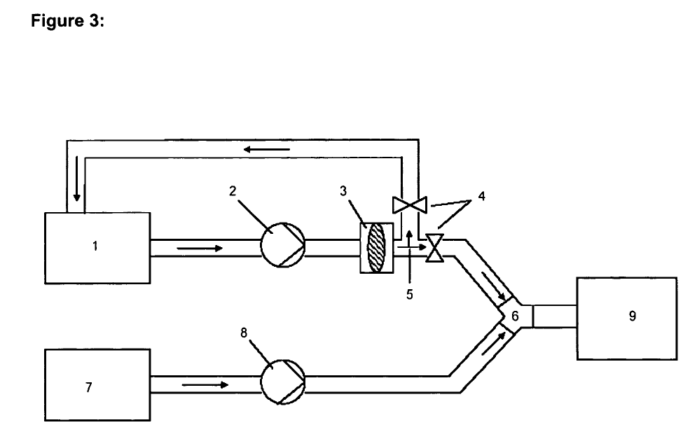

According to another preferred embodiment of the present invention this “combined” process is carried out in a sealed system under aseptic conditions. An apparatus with which a correspondingly combined process can be carried out is shown by way of example in FIG. 3. Starting from a holding vessel (1) which contains the lipid suspension, the lipid suspension is pumped continuously by means of a pump (2) through an extrusion apparatus with a porous membrane of 600-900 nm (3). After the extrusion apparatus is a branch (5) which on the one hand allows the lipid suspension to be refluxed into the holding vessel (1), and thus on the one hand enables the lipid suspension to be extruded several times or alternatively allows the extruded lipid suspension to be conveyed to the Y-shaped member (6) through which the mixing with the nucleic acid takes place. Between the branch (5) and the holding vessel (1) as well as between the branch (5) and the Y-shaped member (6) there are valves (4), through which the refluxing or flow towards the Y-shaped member can be regulated. In a second holding vessel (7) is the sterile-filtered nucleic acid which can be pumped directly to the Y-shaped member (6) through a second pump (8). The lipoplexes formed by mixing extruded lipid suspension and nucleic acid may be collected in a collecting container (9). Preferably, the apparatus is a sealed system, so that the manufacturing process can be carried out under aseptic conditions. A corresponding apparatus is also an object of the present invention.

Using the process according to the invention described here it was surprisingly possible to obtain the high degree of homogeneity which the liposomes had as a result of the special extrusion process, after complexing with the nucleic acid as well. The lipoplex mixtures prepared within the scope of the present invention were characterised by lipoplexes measuring 250-600 nm, preferably 275-500 nm, more preferably 275-400 nm and by a low polydispersity index of ≦0.5, preferably ≦0.4 and in some cases even ≦0.3. Thanks to the high degree of automation it was possible to produce homogeneous lipoplex mixtures spanning more than one batch, which are particularly suitable as pharmaceutical compositions(n) for use in gene therapy or for preparing such compositions. Consequently according to another embodiment the present invention also relates to lipoplex mixtures consisting of lipoplexes with a defined size of between 250 and 600 nm, the lipoplexes consisting of a mixture of homogeneous liposomes according to the invention, as described above, and nucleic acid molecules, characterised in that the polydispersity index of the lipoplex mixture has a value of ≦0.5, preferably ≦0.4. In a preferred embodiment, the lipoplexes are corresponding liposome-nucleic acid complexes which consist of DC30- or preferably DAC30-nucleic acid complexes with corresponding physical parameters.

In another aspect the present invention relates to a process for lyophilising the lipoplexes according to the invention described here, preferably lipoplexes containing a mixture of DOPE and DC-Chol or DOPE and DAC-Chol, preferably in the ratio 70:30 (DC30 or DAC30). Using the process described below it is possible to store the lipoplexes for longer periods, preferably at least 8 months (cf. Table 20). As shown in the Examples, once reconstituted the lipoplexes do not differ from non-lyophilised (i.e. “freshly” prepared) lipoplexes either in their physical parameters or in their bioactivity. The process according to the invention for lyophilising lipoplexes is carried out in the presence of a suitable stabiliser, predominantly in the presence of 250 mM sucrose and 25 mM sodium chloride, and comprises the following steps: (a) freezing the lipoplex mixture to a temperature of ≦−50° C.; (b) drying the lipoplex mixture at approximately −20° C. for at least 35 hours, preferably in vacuo for 35-60 hrs. (c) drying the lipoplex mixture at approximately 20° C. for at least 10 hours, preferably for 10-24 hrs. The times are to be regarded as a guide.

The stabilisers used may be for example various sugars, sugar alcohols or polymers. These may be used as individual components, as a mixture and/or in conjunction with salts. Corresponding examples of suitable sugars, sugar alcohols, polymers and salts are given above. It is advantageous to use the stabiliser in the form of an isoomotic solution (about 290-330 mOsm), preferably in the preparation of the liposomes. The lipids may for example be suspended before extrusion in a corresponding solution which contains a corresponding stabilising agent. However, it is also conceivable to stabilise the lipoplexes by adding the stabilising agent during the lipoplex production, for example by taking up the nucleic acid in a solution which contains a corresponding stabilising agent. It is particularly advantageous for these purposes to use a composition which contains saccharose as the disaccharides and sodium chloride as an inorganic salt. One example of an isoosmotic composition of this kind (e.g. 300 mOsm) is a combination of sodium chloride in a concentration within the range from about 5 mM to about 100 mM, particularly 5, 10, 15, 20, 25, 30, 35, 40, 45 or 50 mM, with a corresponding proportion of saccharose. It is also preferred, for the above purposes, to use a composition containing mannitol on its own or combined with at least one other mono- and/or disaccharide such as e.g. saccharose or trehalose. For example, an isoosmotic composition of this kind (e.g. 300 mOsm) may contain a combination of mannitol in a concentration within the range from about 10-290 mM, particularly about 150-290 mM, and saccharose or trehalose accordingly in a concentration within the range from about 10-290 mM, particularly about 10-150 mM. In this context it has proved advantageous to use saccharose together with sodium chloride, preferably 250 mM saccharose and 25 mM sodium chloride.

It has proved particularly advantageous to lyophilise the lipoplexes described above using a process which comprises the following steps: (a) freezing the lipoplex mixture to a temperature of ≦−50° C. at a temperature lowering rate of approximately ≦1° C./min; (b) incubating the lipoplex mixture at ≦−5-50° C. for at least 2 hours; (c) heating the lipoplex mixture to approximately −20° C. at a heating rate of approximately ≦0.3° C./min; (d) drying the lipoplex mixture at approximately −20° C. for at least 35 hrs, preferably for 35-60 hrs; (e) heating the lipoplex mixture from about −20° C. to about 20° C. at a heating rate of approximately ≦0.44° C./min; (f) drying the lipoplex mixture at about 20° C. for at least 10 hrs, preferably 10-24 hrs. Consequently the present invention also relates to a corresponding process. During the drying (step (d)) pressures of between 0.01-0.1 mbar, preferably between 0.025-0.05 mbar have proved particularly advantageous (cf. Examples, Tables 11 and 13).

Moreover, the present invention also relates to lipoplex lyophilisates which are prepared by one of the processes described here.

The present invention further comprises the use of the lipoplex mixtures according to the invention, directly or in lyophilised form, in gene therapy including combined therapy with pharmacological active substances. It may be useful to combine gene therapy with other therapeutic approaches, such as e.g. the administration of pharmacological active substances, including proteins and/or peptides.

Usually, the lipoplexes or a pharmaceutical composition containing them is or are administered in a total dose within the range from about 0.1 to about 40 μg (including all the values in between), based on the total amount of nucleic acid. In this context it is clear to the skilled man that the phrase “values in between” denotes all the values between the upper and lower limits specified, such as 0.2, 0.3, 0.4, 0.5, 0.6, 0.7, etc.; 1.0, 1.1, 1.2, 1.3, 1.4, 1.5, etc., 2.0, 2.1, 2.2, 2.3, 2.4, 2.5, etc.; 3.0, 3.1, 3.2, 3.3, 3.4, 3.5, etc.; 4.0, 4.1, 4.2, 4.3, 4.4, 4.5, etc.; 5.0, etc.; 6.0, etc.; 7.0, etc.; 8.0, etc.; 9.0, etc.; 10.0, etc.; 11.0, etc., 12.0, etc.; 13.0, etc.; 14.0, etc.; 15.0, etc.; 16.0, etc., 17.0, etc.; 18.0, etc.; 19.0, etc.; 20.0, etc.; 25.0, etc.; 30.0, etc.; 35.0, etc.; 40.0. It is obvious that the dose actually used, the exact composition, the time and method of administration and other details of the treatment may be varied. Suitable animal models which may be used are either the normal domestic pig (Schwartz, R. S. et al. (1990), Circulation 82, 2190; Karas, S. P. et al. (1992) J. Am. Coll. Cardiol. 20, 467) or the so-called mini-pig (Tumbleson, M. E. and Schook, L. B. (1996) Advances in swine in biomedical research, Plenum Press, New York, Bd. 2, 684; cf. also Unterberg, C. et al. (1995) J. Am. Coll. Cardiol. 26, 1747). The results obtained in these models can then be transferred to humans accordingly. The pharmaceutical composition is preferably administered in a total dose within the range from about 0.5 μg to about 10 μg, most preferably about 1 μg to about 5 μg, in each case based on the total amount of nucleic acid.

### EXAMPLES OF EMBODIMENTS

The Examples that follow serve as a further illustration of the objects and processes according to the invention.

Material and Methods:

Chemicals and Cells:

The adjuvants used meet the requirements for pharmaceutically permitted adjuvants: saccharose (Südzucker AG, München, DE), sodium chloride (Merck KG, Darmstadt, DE), WFI (water for injection) (Boehringer Ingelheim, Biberach, DE).

All the lipids used are commercially obtainable: DC-Cholesterol and DOPE can be obtained from Avanti Polar Lipid, Inc., DAC30 at G.O.T. Therapeutics Berlin, DE.

The plasmids used, hereinafter also simply referred to as nucleic acids in some cases, such as e.g. pMCP-1, an MCP-1 (monocyte chemoattractant protein 1) coding plasmid, PEGFP, an EGFP (enhanced green fluorescent protein of A. victoria) expressing plasmid, were cloned and prepared by Boehringer Ingelheim. To do this the coding region of MCP-1 or EGFP was cloned behind a heterologous promoter (CMV promoter). The plasmid also comprised a selectable marker gene (neomycin phosphotransferase gene), so that positively transfected cells could be selected in the presence of a selecting agent (e.g. G-418). The plasmids were about 5 kilobase pairs in size. The BHK21, COS-7, HASMC, A-10 SMC cells used for the transfection experiments are obtained from ATCC (American Type Culture Collection) and grown according to the instructions supplied with them.

Preparation of a Binary Lipid Film:

A binary lipid film is produced according to the “Technical Information” instructions provided by Avanti Polar Lipid Inc. The two lipids (cationic lipid and helper lipid DOPE) are dissolved separately in chloroform or a chloroform:methanol mixture (2:1 v/v). Then the two lipids are titrated together in the desired mass ratio. A lipid mixture consisting of 30 w % DC-cholesterol and 70 w % DOPE is hereinafter referred to as DC30 (the number after the abbreviation of the cationic lipid indicates it proportion by mass in the mixture). For the general process cf. also Pleyer et al. Exp. Eye Res. (2001) 73: 1-7). A lipid mixture consisting of 30 w % DAC-cholesterol and 70 w % DOPE is hereinafter referred to as DAC30. The lipid mixture is then filtered sterile. The binary lipid mixture dissolved in the organic solvent is transferred into a freeze-drying apparatus which has been pre-cooled to −20° C. and the sample is equilibrated until the temperature of the solution is in equilibrium. The solvent is eliminated overnight at −20° C. at a pressure of 0.94 mbar. Then the residual traces of organic solvents are eliminated under a high vacuum (10⁻³ mbar). Alternatively, the organic solvent may be eliminated by blowing a nitrogen or argon current through the sample (shaking gently) and heating the sample to about 30-40° C. The residual traces of organic solvents are also eliminated under a high vacuum. These operations are carried out under aseptic conditions. Lipid suspensions may then be prepared from the lipid films thus obtained.

Preparation of a DC30 Suspension:

To prepare a 1 mg/ml lipid suspension of DOPE/DC-Chol 70/30 (w/w), 1 ml of transfection solution (250 mM saccharose, 25 mM NaCl) and 1 mg of DC30 are mixed and left to swell for 30 min at ambient temperature. From this a 0.25 mg/ml DC30 lipid suspension is prepared by diluting accordingly with transfection solution.

Preparation of a DAC30 Suspension:

To prepare a 1 mg/ml lipid suspension of DOPE/DAC-Chol 70/30 (w/w), 1 ml of transfection solution (250 mM saccharose, 25 mM NaCl) and 1 mg of DAC30 are mixed and left to swell for 30 min at ambient temperature. From this a 0.25 mg/ml DAC30 lipid suspension is prepared by diluting accordingly with transfection solution.

Thawing, Dissolving and Sterile Filtration of the Nucleic Acid:

The nucleic acid (1 mg/ml) stored at −20° C. is thawed in the refrigerator at 2-8° C. and diluted to the desired concentration with transfection solution (250 mM saccharose, 25 mM NaCl). At the same time the plasmid (for example pMCP-1 or pEGFP) is stirred into the transfection solution (0.05 mg/ml). Then it is filtered sterile through a 0.2 μm sterile filter (different sizes of sterile filter are used, depending on the amount to be filtered: e.g. Millipak TM 20:100 cm² filter surface, Millipak TM 40:200 cm² filter surface, Messrs. Millipore). The filtration is done using a peristaltic pump. Before and after the sterile filtration an integration test is carried out on the filter (bubble point method, nominal bubble point: 3.45 mbar, according to Pharm. Eu and according to “GMP-Berater”, reference work for the pharmaceutical industry and suppliers, October 2001, GMP Verlag).

Determining the Particle Size and the Polydispersity Index

The particle size (given as the mean diameter Ø) and also the polydispersity index (PI) of the liposomes and lipoplexes is determined by PCS (Photon Correlation Spectroscopy) (apparatus: Malvern Zetasizer 3000 HS, Malvern Autosizer 4700, Malvern Instrument Ltd., Worcestershire, UK, Nicom 380, Nicom Technologies INC, USA). All the instruments were calibrated using the same latex standard. At the same time the scattering of an He-Ne laser is measured on the sample at an angle of 90° (according to ISO 13321: 1996(E)) and the two parameters (Ø and PI) are determined from the scattering data by evaluation with the cumulant analysis. The breadth of the particle distribution is described by the dimensionless polydispersity index (PI) and is defined according to ISO 13321: 1996(E) as follows:
PI=μ₂/ [symbol] Γ [symbol] ²=σ²/2 [symbol] Γ [symbol] ²

With [symbol] Γ [symbol] average rate of decay, μ₂=∫(Γ− [symbol] Γ [symbol] )² G(Γ)dΓ,
σ=standard deviation (for more precise information see ISO 13321: 1996(E))

The mean particle diameter x_{PCS} (hereinafter referred to only as Ø) is defined according to ISO 13321: 1996(E) as:
1/x_{PCS}=∫(1/x)G[Γ(x)]d(1/x).
Lipid Analysis by HPLC (High Performance Liquid Chromatography):

The lipid concentration and also the ratio of cationic lipid (e.g. DC-Chol) to helper lipid (e.g. DOPE) is determined by HPLC (High Performance Liquid Chromatography). Cf. on this subject Chang C. D & Harris D. J. (1998) J. Liqu. Chrom. & Rel. Technol. V21: 1119-1136 or Meyer O., et al. (2000) Eur. J. Pharm. Biopharm. 50: 353-356.

Nucleic Acid Analysis:

The quality of the nucleic acid used based on the evaluation of ccc, oc, and linear proportion is determined using agarose gel (ethidium bromide staining) according to general methods (cf. Ausubel, F. M. et al., Current protocols in molecular biology. New York: Green Publishing Associates and Wiley-Interscience. 1994 (updated), Sambrook et al., Molecular Cloning: A Laboratory Manual, 2nd Ed., Cold Spring Harbor Laboratory Press, Cold Spring Harbor, N.Y., 1989). The content is determined by PicoGreen Assay or UV spectroscopy.

Karl-Fischer Titration:

The residual moisture content in samples of lyophilisate is determined by the Karl Fischer method (European Pharmacopoeia, 2002).

Determining the Transfection Efficiency:

The protein expression of the transfected cells is determined using a commercially obtainable kit in accordance with the instructions provided by the kit manufacturer: MCP-1: Human MCP-1 ELISA Kit made by BD Biosciences Pharmingen, BD Biosciences, San Diego, USA. The expression of EGFP is determined using a FACS apparatus (fluorescence activated cell sorter) made by Messrs Becton Dickinson BD Biosciences, San Jose, USA in accordance with the manufacturer's instructions (filter 488 nm).

### Example 1

### Preparation of homogeneous liposomes DOPE/DC-Chol 70/30 (DC30)

Influence of the Extrusion Time/Cycles on the Size and Homogeneity of the Liposomes:

In order to produce homogeneous DOPE/DC-Chol 70/30 liposomes a DC30 lipid suspension in transfection medium with a lipid concentration of 1 mg/ml was prepared (see above). By means of a holding vessel the lipid suspension was continuously pumped through a polycarbonate membrane with a pore size of 600 nm (Messrs. Millipore, Billerica, Mass. USA) and a flow rate of 80 ml/min through the sealed low pressure extrusion apparatus (see FIG. 1). The pressure measured at the membrane was less than 1×10⁵ Pa (10⁵ Pa=1 bar). In a corresponding experimental set-up an extrusion time of about 10 min corresponded to approximately 2 extrusion cycles.

| | |
|---|---|
| Apparatus: | Filtron peristaltic pump |
| | Silicon tube (internal diameter 4 mm) |
| Extrusion unit: | Millipore filter housing |
| | 600 nm polycarbonate membrane/Ø 47 mm |
| Holding vessel: | 1000 ml separating funnel |

As can be seen from Table 1, the size of the liposomes is controlled by the number of extrusions carried out (extrusion cycles). Non-extruded liposomes have an average size of more than 1500 nm, while the scattering, given as the ± standard deviation (SD) in relation to the average liposome size, is about ±80%. As the number of extrusion cycles increases, liposomes with an average size of approximately 600 to 300 nm are formed, while the homogeneity of the liposomes increases as the number of extrusions increases (cf. Table 1).

TABLE 1

| time | size | ±SD | ±SD | pressure | |
|---|---|---|---|---|---|
| (min) | (nm) | (nm) | (%) | (bar) | cycles |
| 0 | 1606 | 1283 | 80 | — | 0.0 |
| 0 | 1575 | 1362 | 86 | — | 0.0 |
| 10 | 607 | 371 | 61 | 0.6 | 2.1 |
| 10 | 606 | 374 | 62 | 1 | 2.1 |
| 15 | 535 | 293 | 55 | 0.6 | 3.2 |
| 15 | 500 | 270 | 54 | 1 | 3.2 |
| 20 | 477 | 269 | 56 | 0.6 | 4.3 |
| 20 | 441 | 247 | 56 | 1 | 4.3 |
| 25 | 435 | 226 | 52 | 0.6 | 5.3 |
| 25 | 407 | 210 | 52 | 1 | 5.3 |
| 30 | 411 | 214 | 52 | 0.6 | 6.4 |
| 30 | 394 | 190 | 48 | 0.9 | 6.4 |
| 35 | 379 | 188 | 50 | 0.6 | 7.5 |
| 40 | 377 | 180 | 48 | 0.6 | 8.5 |
| 45 | 345 | 172 | 50 | 0.9 | 9.6 |

Effect of Different Flow Rates on the Size and Homogeneity of the Liposomes:

The process was carried out analogously to the Example described above. The flow rates were selected so that the pressure on the membrane does not exceed a value of 3×10⁵ Pa. The flow rates were 210, 110, 54 ml/min. The extrusion volume was 100 ml per mixture in each case. All the other process parameters were adjusted as described above. As can be seen from Table 2, the flow rate has a considerable influence on the resulting size distribution and homogeneity of the liposomes. A reduction in the flow rate to less than 100 ml/min over the same extrusion period produces larger liposomes.

TABLE 2

| | flow rate | | | | | |
|---|---|---|---|---|---|---|
| | 210 ml/min | | 110 ml/min | | 54 ml/min | |
| time (min) | size ± SD | | size ± SD | | size ± SD | |
| 0 | 1626 | nm (1270) | 1804 | nm (1360) | 1573 | nm (1134) |
| 2 | 423 | nm (186) | 557 | nm (316) | 689 | nm (380) |
| 5 | 371 | nm (163) | 429 | nm (212) | 522 | nm (278) |
| 10 | 358 | nm (163) | 374 | nm (177) | 436 | nm (198) |

Effect of Pore Size, Lipid Concentration and Number of Extrusion Cycles on the Size and Homogeneity of the Liposomes:

In the preceding Example the extrusion was carried out with a 600 nm and an 800 nm polycarbonate membrane. To do this, DC30 lipid suspensions with a lipid concentration of 1 mg/ml or 0.25 mg/ml were prepared as described above and by means of a holding vessel pumped continuously through a polycarbonate extrusion membrane with a corresponding pore size of 600 nm or 800 nm (Millipore, supra) and a flow rate of 80 ml/min. The pressure measured at the membrane was less than 1×10⁵ Pa in each case. The description of the samples can be found in Table 3. Sample 7 was prepared by diluting sample 6, which had previously been the extruded three times, to 0.25 mg/ml with transfection solution.

TABLE 3

| sample | | |
|---|---|---|
| number | sample/concentration | Remark |
| 1 | DC30 lipid, saccharose-NaCl solution | 1 passage |
| | conc. 0.25 mg/mL | |
| 2 | DC30 lipid, saccharose-NaCl solution | 2 passages |
| | conc. 0.25 mg/mL | |
| 3 | DC30 lipid, saccharose-NaCl solution | 3 passages |
| | conc. 0.25 mg/mL | |
| 4 | DC30 liposomes, | 1 passage |
| | conc. 1 mg/mL | |
| 5 | DC30 liposomes, | 2 passages |
| | conc. 1 mg/mL | |
| 6 | DC30 liposomes, | 3 passages |
| | conc. 1 mg/mL | |
| 7 | Probe 6 after 3 extrusions with | dilution |
| | saccharose-NaCl solution diluted to | |
| | 0.25 mg/mL | |

The above experiments show that the size of the liposomes can be adjusted precisely, depending on the choice of the pore size of the membrane, extrusion cycles and lipid concentration. Three extrusions of a 0.25 mg/ml lipid dispersion give different results, depending on the pore size of the extrusion membrane. When a 600 nm membrane was used the liposome size was 281 nm. When an 800 nm membrane was used the liposome size was 352 nm. If on the other hand higher lipid concentrations are used, after 3 passages similar diameter values are obtained after dilution of the liposomes. These tests show that the three process parameters of lipid concentration, extrusion cycles (time) and pore size of the extrusion membrane have to be precisely adjusted to one another. As the number of extrusions increases, liposomes with a homogeneous size distribution can be produced (cf. Table 4). The polydispersity index of the extruded liposomes decreased with the number of extrusions. The extrusion of a DC30 lipid suspension with 0.25 mg/ml resulted in more homogeneous liposomes compared to extruded DC30 lipid suspensions with a lipid concentration of 1 mg/ml.

TABLE 4

| | | 600 nm membrane | | 800 nm membrane | |
|---|---|---|---|---|---|
| | | size | Poly- | size | Poly- |
| liposome sample | | (nm) | Index | (nm) | Index |
| sample 1 | 1× extruded | 348 | 0.53 | 420 | 0.55 |
| sample 2 | 2× extruded | 290 | 0.40 | 366 | 0.54 |
| sample 3 | 3× extruded | 281 | 0.30 | 352 | 0.45 |
| sample 4 | 1× extruded | 465 | 0.51 | 430 | 0.65 |
| sample 5 | 2× extruded | 390 | 0.56 | 372 | 0.67 |
| sample 6 | 3× extruded | 380 | 0.59 | 355 | 0.57 |
| sample 7 | dilution | 369 | 0.42 | 368 | 0.46 |

Stability of the Extruded Liposomes:

The liposomes prepared by continuous low pressure extrusion (concentration 1 mg/ml, flow rate 80 ml/min, pressure less than 1×10⁵ Pa, 600 nm membrane) were stored at ambient temperature and at 4° C. The effects of storage on the stability (=size) of the liposomes at different times were measured.

TABLE 5

| | particle size in nm | | | | | | |
|---|---|---|---|---|---|---|---|
| extrusion | | 6 h | | 12 h | | 24 h | |
| time/min | instantly | RT | 4° C. | RT | 4° C. | RT | 4° C. |
| 5 | 478 | 489 | 482 | 463 | 471 | 487 | 482 |
| 10 | 397 | 389 | 392 | 386 | 395 | 379 | 385 |
| 15 | 368 | 372 | 379 | 375 | 368 | 381 | 375 |
| 30 | 352 | 348 | 346 | 356 | 351 | 360 | 354 |

It will be seen that the size of the liposomes remains stable over 24 hours at 4° C. and at ambient temperature. Thus, no “frustrations” (surface tensions) are built into the liposomes which would lead to fusion. The experiments carried out and the preceding Example show that: Continuous flow extrusion at low pressure (less than 3·10⁵ Pa=3 bar) is a suitable method of reproducibly preparing liposomes ranging in size between about 250 and 800 nm. The size of the liposomes can be adjusted by the extrusion time, the flow rate, and the pore size of the extrusion membrane. The size of the liposomes remains stable over a period of at least 24 hours both at ambient temperature and at 4° C. and thus allows reliable and simple “further processing” of the liposomes to form lipoplexes. The process can easily be adapted for use on an industrial scale in the manner of a smoothly “scaleable” and aseptic validatable process. During the extrusion process (600 nm extrusion membrane) the quality of the DC30 liposomes remains unchanged. Neither the concentration nor the ratio of DC-Chol to DOPE is altered. The lipid content of the liposomes (Table 6) was determined by HPLC (high performance liquid chromatography).

TABLE 6

| | sample | | | |
|---|---|---|---|---|
| | | batch 1 | batch 2 | batch 3 |
| | composition | 1 extrusion | 2 extrusions | 3 extrusions |
| | (intended) | (μg/mL) | (μg/mL) | (μg/mL) |
| liposomes | DC-Chol | 33.4 | 30.8 | 31.4 |
| after extrusion | 30 μg/mL | | | |
| | DOPE | 72.1 | 68.3 | 69.7 |
| | 70 μg/mL | | | |

### Example 2

### Preparation of Homogeneous Liposomes DOPE/DAC-Chol 70/30 (DAC30)

On the basis of the findings from Example 1 homogeneous liposomes with a size between 250 and 800 nm and a polydispersity index of ≦0.6 were prepared. In order to prepare homogeneous liposomes DOPE/DAC-Chol 70/30 w/w (DAC30) a DAC30 lipid film was incubated for 30 min with transfection medium (250 mM saccharose, 25 mM NaCl) and left to swell for 30 min. The lipid concentration was adjusted to 1 mg/mL. The lipid suspension was transferred into the low pressure extrusion apparatus. Then the lipid suspension was pumped continuously and evenly (analogously to Example 1) through a polycarbonate membrane with a pore size of 800 nm (Messrs. Millipore, Billerica, Mass. USA) at a flow rate of 80 ml/min through the sealed low pressure extrusion apparatus (see FIG. 1). The pressure measured on the membrane was less than 1×10⁵ Pa. In a corresponding experimental set-up an extrusion time of 5 min corresponded to one (1) extrusion cycle. The experiment was carried out under aseptic conditions with sterile materials and apparatus.

| | |
|---|---|
| apparatus: | Filtron peristaltic pump |
| | Silicon tube (internal diameter 4 mm) |
| extrusion unit: | extrusion housing Gelman Sciences 2220 |
| | 800 nm polycarbonate membrane/Ø 47 mm |
| holding vessel: | 1000 ml separating funnel |

Then the extruded DAC30 liposomes were diluted with sterile-filtered transfection medium (250 mM saccharose, 25 mM NaCl) to a lipid concentration of 0.25 mg/ml.

After swelling of the lipid film, liposomes measuring from 500 to 1500 nm are obtained, the size of which generally cannot be reproduced from batch to batch (cf. Table 7). After a single extrusion of the 1 mg/mL lipid suspension through the 800 nm polycarbonate membrane, DAC30 liposomes were obtained measuring from 430 to 450 nm with a narrow polydispersity index (=PI) in the range from 0.4 to 0.5. The size of the liposomes after dilution of the 1 mg/ml extruded liposomes to a final concentration of 0.25 mg/ml was in the range from 420-440 nm (PI=0.4 to 0.5).

TABLE 7

| | | | After extrusion | | After dilution | |
|---|---|---|---|---|---|---|
| | Before extrusion | | 1 mg/ml | | 0.25 mg/ml | |
| Experiment | size (PI) | | size (PI) | | size (PI) | |
| 1 | 1100 | nm (0.99) | 447 | nm (0.47) | 439 | nm (0.40) |
| 2 | 502 | nm (0.96) | 434 | nm (0.45) | 423 | nm (0.49) |

The liposomes thus prepared may be stored for several days (7 days) at 2-8° C. without losing their quality. After 7 days' storage at 2-8° C. the size of the liposomes (experiment 2 from Table 7, 0.25 mg/mL) was 425 nm (PI=0.48).

### Example 3

### Preparation of Homogeneous Lipoplexes Consisting of DOPE/DC-Chol 70/30 (DC30) and Nucleic Acid

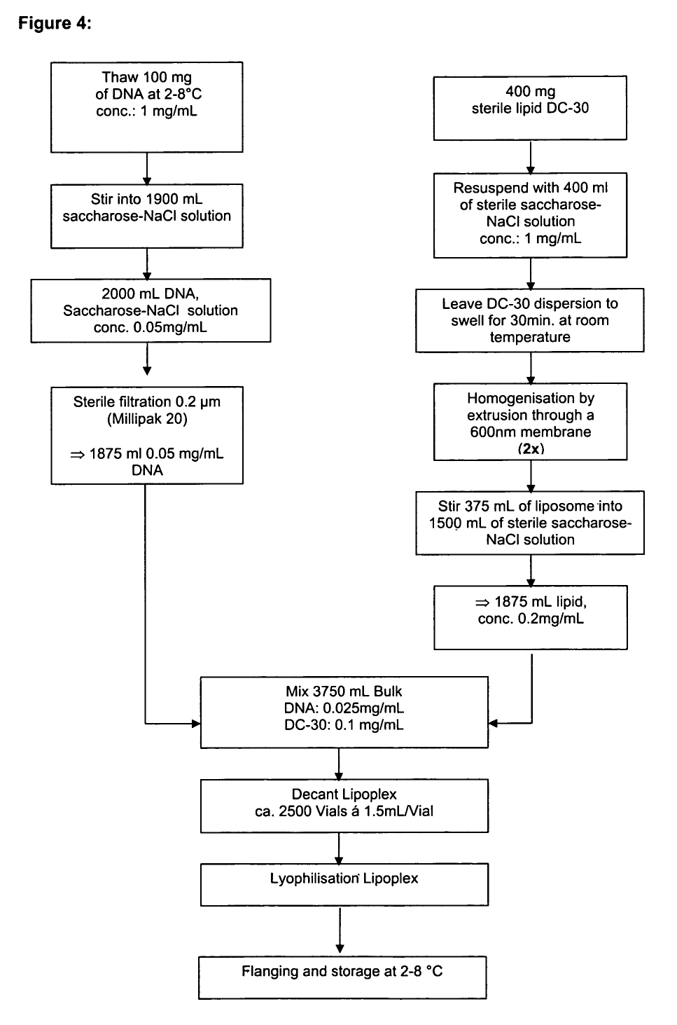

On the basis of the findings from Example 1 homogeneous liposomes were prepared. The flow diagram (FIG. 4) shows the individual steps in the preparation of the DC30 lipoplexes. In the preceding example the mass ratio of lipid to DNA was 4:1, which corresponds to a charge ratio +/− of about 0.75. 400 mg of sterile lipid DC30 were combined with 400 ml of sterile transfection solution (250 mM saccharose and 25 mM NaCl), so that the lipid concentration was 1 mg/ml. After 30 min. swelling at ambient temperature extrusion was carried out twice through a 600 nm polycarbonate membrane. 375 ml of this lipid suspension were combined with 1500 ml of transfection solution. 1875 ml of a 0.2 mg/mL DC30 lipid suspension were obtained. The liposomes measured 310 nm (PI=0.35).

DNA (100 mg) in a concentration of 1 mg/ml, thawed at 2-8° C., was stirred into 1900 ml of transfection solution. The DNA concentration after the dilution step was 0.05 mg/mL. The DNA solution was then filtered sterile. 1875 ml of this sterile-filtered DNA solution was mixed with the DC30 liposomes in the next step. The starting volumes of the liposome suspension and DNA solution were 1875 ml in each case and the concentrations of the liposome suspension (0.2 mg/mL) and DNA solution (0.05 mg/mL) were adjusted so that the mass ratio of lipid to DNA is 4:1.

Stable lipoplexes are obtained by carrying out the process illustrated in FIGS. 3 and 4. The preceding mixing process must be carried out so as to ensure uniform and continuous combining of the liposomes and nucleic acids. For this purpose the liposome suspension and DNA solution were evenly and continuously mixed through a Y-shaped member (e.g. Hibiki Y-1Ø3 mm, Hibiki Y-2Ø5 mm for larger volumes, or Norma Y-shaped member Ø3 mm and Ø5 mm) and transferred into a sterile flask. Other geometric arrangements are also possible. The Y-shaped member used had a diameter of 3 mm (FIG. 3). The Y-shaped member may be made of polypropylene (PP), polyethylene (PE) or polyvinyl chloride (PVC) or from stainless steel or glass. The two pumps for the nucleic acid solution and for the liposome suspension have to be “switched the same” (=identical flow rates), so that the solutions can be pumped evenly and continuously. If a single pump is used which enables 2 liquids to be conveyed separately, care must be taken to ensure that the two liquids are sucked in simultaneously and combined and mixed through the Y-shaped member. The throughflow rate in the preceding example was 150-170 ml/min. The volumes of the two starting solutions were the same.
apparatus: Ismatec peristaltic pump
Y-shaped member: Ø am, Hibiki Y-1 (Carl Roth GmbH & Co. KG., Karlsruhe, DE)
2 silicon tubes Messrs. ITE (Ø 4 mm) (Intertechnik Elze, Elze, DE)

After mixing the lipoplex bulk (3750 ml) was left to stand for at least 30 min at ambient temperature before being further processed: concentration of DNA: 0.025 mg/mL; concentration of lipid: 0.1 mg/ml.

After 30 min after the preparation of the lipoplexes the lipoplex size was generally 270-310 nm and hardly changed over 3 hrs' storage at ambient temperature (Table 8).

TABLE 8

| | Experiment | | | |
|---|---|---|---|---|
| | 1 | | 2 | |
| | size (nm) | Poly-Index | size (nm) | Poly-Index |
| After 0.5 hrs. | 297 | 0.15 | 289 | 0.17 |
| After 3 hrs. | 275 | 0.15 | 278 | 0.15 |

The next Table (Table 9) shows PCS results (measured after 30 min) of liposomes (DC30) and lipoplexes (DC30/DNA 4:1 w/w) which were prepared by the mixing process described above. The liposome suspension was obtained by various extrusion cycles (600 nm extrusion membrane). It will be seen that the starting conditions affect the quality of the lipoplexes. The data in Tables 8 and 9 show that extruding the liposomes twice through the same extrusion membrane yields liposomes with which lipoplexes can be produced in a size range of from 280-310 nm with PI<0.3.

TABLE 9

| | sample | | | | | |
|---|---|---|---|---|---|---|
| | batch 1 | | batch 2 | | batch 3 | |
| | 1 extrusion | | 2 extrusions | | 1 extrusion | |
| | size | Poly- | size | Poly- | size | Poly- |
| | (nm) | Index | (nm) | Index | (nm) | Index |
| liposomes after | 414 | 0.43 | 338 | 0.35 | 323 | 0.31 |
| extrusion | | | | | | |
| lipoplex before | 338 | 0.30 | 281 | 0.20 | 269 | 0.16 |
| lyophilisation | | | | | | |
| lipoplex after | 338 | 0.31 | 284 | 0.22 | 259 | 0.18 |
| lyophilisation | | | | | | |

### EXAMPLE 4

### Preparation of Homogeneous Lipoplexes Consisting of DOPE/DAC-Chol 70/30 (DAC30) and Nucleic Acid

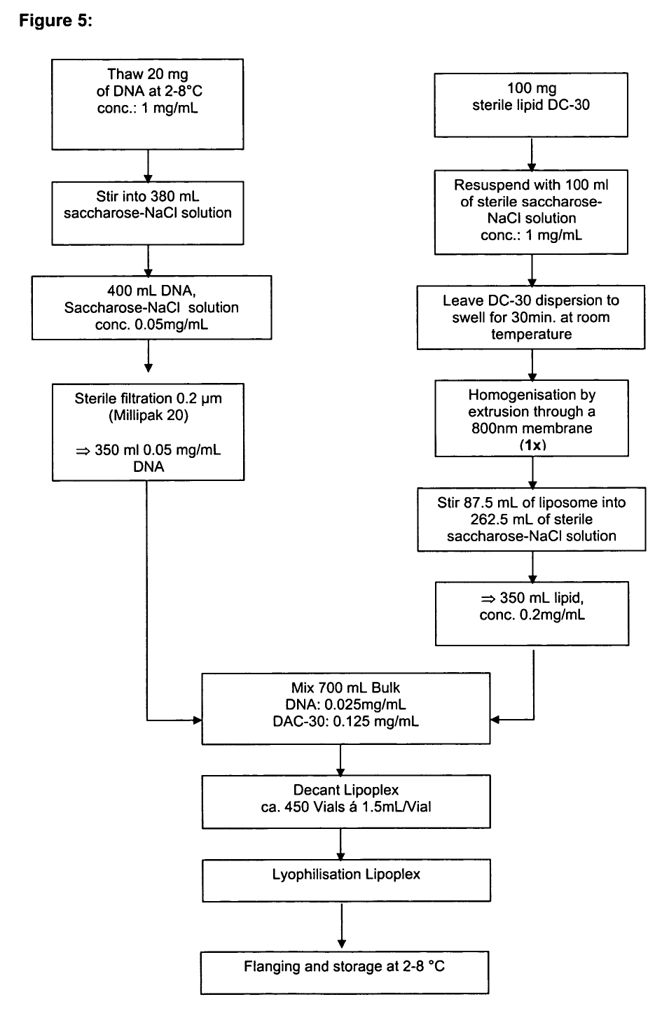

On the basis of the findings from Example 2 homogeneous liposomes were prepared. The flow diagram (FIG. 5) shows the individual steps in the preparation of the DAC30 lipoplexes. In the preceding example the mass ratio of lipid to DNA was 5:1, which corresponds to a positive to negative charge ratio+/−of about 1. For this, 100 mg of sterile lipid DAC30 were combined with 100 ml of sterile transfection solution (250 mM saccharose and 25 mM NaCl), so that the lipid concentration was 1 mg/ml. After 30 min. swelling at ambient temperature extrusion was carried out once through an 800 nm polycarbonate membrane. 87.5 ml of this lipid suspension were combined with 262.5 ml of transfection solution so as to obtain 350 ml of a 0.25 mg/mL DC30 lipid suspension. The liposomes measured about 430 nm (PI=0.4).

DNA (20 mg) in a concentration of 1 mg/ml, thawed at 2-8° C., was stirred into 380 ml of transfection solution. The DNA concentration after the dilution step was 0.05 mg/mL. The DNA solution was then filtered sterile. This sterile-filtered DNA solution was mixed with the DAC30 liposomes in the next step. The starting volumes of the liposome suspension and DNA solution were 350 ml in each case. The concentrations of the liposome suspension (0.25 mg/mL) and DNA solution (0.05 mg/mL) were adjusted so that the mass ratio of lipid to DNA is 5:1. Stable lipoplexes are obtained by carrying out the process illustrated in FIGS. 3 and 5. The mixing process must be carried out so as to ensure uniform and continuous combining of the liposomes and nucleic acids (see Example 3). The liposome suspension and DNA solution were evenly and continuously mixed through a Y-shaped member (FIG. 9) and transferred into a sterile flask. The Y-shaped member used had a diameter of 3 mm. The two pumps for the nucleic acid solution and for the liposome suspension were “switched the same, so that the solutions can be pumped evenly and continuously. If a single pump is used which enables 2 liquids to be conveyed separately, care must be taken to ensure that the two liquids are sucked in simultaneously and combined and mixed through the Y-shaped member. The throughflow rate in the preceding example was 400 ml/min. The volumes of the two starting solutions were the same.

apparatus: Ismatec peristaltic pump

Y-shaped member: Ø3 mm, Hibiki Y-1 (see above)
2 silicon tubes Messrs. ITE (Ø4 mm)

After mixing the lipoplex bulk (700 ml) was left to stand for at least 30 min at ambient temperature before being further processed: concentration of DNA: 0.025 mg/mL; concentration of lipid: 0.125 mg/ml.

The quality of the lipoplexes prepared according to Example 4 is summarised in Table 10. This shows the PCS results from 4 different batches which were prepared by the process described.

TABLE 10

| batch | size (nm) | Poly-Index |
|---|---|---|
| 1 | 309 | 0.27 |
| 2 | 300 | 0.28 |
| 3 | 312 | 0.24 |
| 4 | 320 | 0.23 |

These Examples clearly show that reproducible batches can be prepared using the process described. The lipoplexes are in the range from 280-330 nm with PI<0.4.

### Example 5

### Packaging of Homogeneous Lipoplexes Consisting of DOPE/DC-Chol 70/30 (DC30) and Nucleic Acid

The DC30 lipoplexes prepared in Example 3 were packaged using a standard filling machine (Messrs. Bausch & Ströble, GmbH & Co. KG., Ilshofen, DE) in accordance with the piston pump filling method into a 2 ml vial (contents 1.5 ml, cf. FIG. 4). This step was also carried out using sterile materials under aseptic conditions. The primary packaging used was:
Vial: 2R colourless injection vial GAl BB (Messrs. Schott, Mainz, DE)
Stopper: Gusto 13 mm V2 F210 3WRS D713
Knurled cap: Kombika/Alu 13 mm (in each case made by The West Company, Germany GmbH, Eschweiler, DE)

### Example 6

### Packaging of homogeneous lipoplexes consisting of DOPE/DAC-Chol 70/30 (DAC30) and nucleic acid

The DAC30 lipoplexes prepared in Example 4 were packaged using a standard filling machine (Messrs. Bausch & Ströble, supra) in accordance with the piston pump filling method. Alternatively, for small bulk volumes, the containers can be filled by hand using Eppendorf pipettes. This step is also carried out using sterile materials under aseptic conditions (packaging as in Example 5).

### Example 7

### Lyophilisation of Homogeneous Lipoplexes Consisting of DOPE/DC-Chol 70/30 (DC30) and Nucleic Acid

Preliminary Tests for the Lyophilisation:

The process of controlling lyophilisation has a considerable influence on the quality of the lyophilisates. The tests on lyophilising lipoplexes were carried out in the Lyo Corn 4018 freeze drying apparatus (lyophiliser) (Messrs. Hof). The technical details of the freeze drying apparatus are summarised in Table 11. The start data for controlling the lyophilisation (start values) are shown in Table 12. The starting process lasts for 53.5 hours in all.

TABLE 11

| | |
|---|---|
| Equipment names | GFT apparatus |
| Model and manufacturer | Lyo Com 4018 (Tiny Lyo) |
| | Hof GmbH 35102 Lohra |
| total shelf space | 0.36 m² |
| number of shelves | 3 |
| position of condenser | external, underneath the drying |
| | chamber |
| max. ice capacity of condenser | 10-15 kg |
| lowest shelf temperature | −55° C. |
| lowest condenser temperature | −80° C. |
| vacuum regulation | Pirani vacuum valve |
| temperature sensor | PT 100 |

TABLE 12

| | | nominal | |
|---|---|---|---|
| | duration (hh:mm) | temperature (° C.) | vacuum (mbar) |
| loading | 00:05 | 5 | 1000 |
| freezing | 00:55 | −50 | 1000 |
| | 00:30 | −50 | 1000 |
| | 01:00 | −50 | 1000 |
| | 00:30 | −50 | 1000 |
| main drying | 01:30 | −20 | 0.05 |
| | 33:30 | −20 | 0.05 |
| | 3:30 | −20 | 0.05 |
| after-drying | 01:30 | 20 | 0.05 |
| | 10:00 | 20 | 0.05 |
| removal | 00:30 | 5 | 0.05 |
| total time | 53:30 | — | |

The lyophiliser was ventilated with nitrogen at the end of the lyophilisation process and the vials were sealed under 600 mbar. Starting from the process parameters shown in Table 12, the changes shown in Table 13 were made to the methods used for tests 1-5 (V01-V05) in order to optimise the process.

TABLE 13

| | | main | after- | changes |
|---|---|---|---|---|
| | | drying | drying | to |
| test | freezing | (MD) | (AD) | programme |
| V01 | from 5 | 0.10 mbar | 0.01 mbar | cooling |
| | to −50° C. | to −30° C. | to 20° C. | gradient |
| | in 30 min | in 30 min | in 1.5 h | to −50° C. |
| | maintain | maintain | maintain | |
| | for 4 h | for 23.5 h | for 4 h | |
| | | to 0° C. | | |
| | | in 1 h | | |
| | | maintain | | |
| | | for 13.5 h | | |
| V02 | from 5 | 0.05 mbar | 0.01 mbar | MD lower |
| | to −50° C. | in 1 h | to 20° C. | vacuum, |
| | in 30 min | to −10° C. | in 1.5 h | increase in |
| | maintain | maintain | maintain | temperature |
| | for 4 h | for 23 h | for 4 h | |
| | | to 0° C. | | |
| | | in 1 h | | |
| | | maintain | | |
| | | for 13.5 h | | |
| V03 | from 5 | 0.025 mbar | 0.01 mbar | freezing |
| | to −50° C. | (actual | to 20° C. | gradient |
| | in 55 min | 0.05-0.025 | in 1.5 h | 1° C./min |
| | maintain | mbar) | maintain | MD extended |
| | for 4 h | to −10° C. | for 4 h | 0.025 mbar |
| | | in 2 h | | vacuum |
| | | maintain | | |
| | | for 36.5 h | | |
| V04 | from 5 | 0.05 mbar | 0.05 mbar | MD at −20° C. |
| | to −50° C. | to −20° C. | to 20° C. | MD and AD |
| | in 55 min | in 1.5 h | in 1.5 h | identical |
| | maintain | 37 h | maintain | vacuum |
| | for 4 h | maintain for | for 4 h | |
| V05 | from 5 | 0.05 mbar | 0.05 mbar | freezing 2 h |
| | to −50° C. | to −20° C. | to 20° C. | (holding phase |
| | in 55 min | in 1.5 h | in 1.5 h | shortened) |
| | maintain | maintain | maintain | variation in |
| | for 2 h | for 37 h | for 8 + 2 h | AD time |

The visual assessment of the lyophilisation cake of the individual lyophilisates is given in Table 14. Visual inspection of the lyophilisation cakes showed that the lyophilisation cake of test V01 was unsuitable as a substantial number of lyophilisation cakes collapsed. These lyophilisation cakes had very long reconstitution times. The residual moisture contents, which were determined by Karl-Fischer titration, are all shown in Table 16. The residual moisture contents of the lyophilisation cakes of test V01 were above 5%.

TABLE 15

| Test | Characteristics of cakes | Evaluation |
|---|---|---|
| V01 | lyo-cakes in the centre of the | unsuitable |
| | part-load have collapsed completely, | |
| | lyo-cakes at the edges have shrunk | |
| | considerably | |
| V02 | all the lyo-cakes have shrunk | suitable (apart from |
| | considerably, a few cakes have | the collapsed cakes) |
| | cavities = collapsed | visually borderline |
| | no clear difference between the | |
| | outside and inside in the tin. | |
| V03 | like V02, ˜10% cakes (distrib- | suitable (apart from |
| | uted over the load) collapsed. | the collapsed cakes) |
| | | visually borderline |
| V04 | cakes shrunk, no difference | suitable |
| | between inside and outside | visually borderline |
| V05 | cakes shrunk, no difference | suitable |
| | between inside and outside | visually borderline |

Lyophilisates which had not collapsed (from V01) exhibited a lower residual moisture content.

Tests V03 and V04 were carried out with different formulations. One formulation contained sodium chloride (250 mM saccharose, 25 mM NaCl), the other did not contain sodium chloride (250 mM saccharose). The data in Table 16 show that the absence of sodium chloride leads to drier lyophilisates. The absence of sodium chloride from the formulation, however, leads to instability of the lipoplex liquid formulation, showing that sodium chloride was necessary. By using longer times for the after-drying it is possible to prepare lyophilisates with residual moisture contents significantly below 3% (V05 from Table 16).

TABLE 16

| | | | | average | Min | Max | | VC |
|---|---|---|---|---|---|---|---|---|
| Test | NT | sample | n | % | % | % | hrs | % |
| V01 | 20° C., | poor | 3 | 559 | 529 | 5.85 | 0.28 | 5 |
| | 4 h | | | | | | | |
| V02 | 20° C., | good | 3 | 320 | 2.61 | 3.57 | 0.52 | 16 |
| | 4 h | (inside) | | | | | | |
| V03 | 20° C., | good | 3 | 274 | 222 | 3.37 | 0.58 | 21 |
| | 4 h | | | | | | | |
| | 20° C., | good/ | 3 | 158 | 1.26 | 1.90 | 0.32 | 20 |
| | 4 h | without | | | | | | |
| | | NaCl | | | | | | |
| V04 | 20° C., | good | 5 | 290 | 2.39 | 3.27 | 0.35 | 12 |
| | 4 h | | | | | | | |
| | 20° C., | good/ | 5 | 132 | 1.13 | 1.47 | 0.13 | 10 |
| | 4 h | without | | | | | | |
| | | NaCl | | | | | | |
| V05 | 20° C., | good | 5 | 223 | 2.02 | 2.74 | 0.29 | 13 |
| | 8 h | | | | | | | |
| | 20° C., | good | 5 | 196 | 1.50 | 2.70 | 0.45 | 23 |
| | 10 h | | | | | | | |

The data for the product temperature of the individual tests are shown in Table 17.

TABLE 17

| | | | sudden | average |
|---|---|---|---|---|
| | | initial MD | drying | temperature |
| test | MD | [° C.] | (° C.) | (° C.) |
| V01 | −30° C./0.10 mbar | −42 | −30 | −36 |
| V02 | −10° C./0.05 mbar | −40 | −18 | −29 |
| V03 | −10° C./0.025 mbar | −40 | −27 | −34 |
| V04 | −20° C./0.05 mbar | −40 | −26 | −33 |
| V05 | −20° C./0.05 mbar | −42 | −30 | −36 |

The glass transition temperature (Tg′) of the two formulations was determined by calorimetry (DSC 821 Messrs. Mettler Toledo (Giessen, DE)):

Tg′ turning point 10° C./min

| | |
|---|---|
| Placebo | −34° C. |
| Placebo without NaCl | −32° C. |
| Verum | −33 to −35° C. |

The main drying should be carried out at a temperature below the glass transition temperature.

To summarise, it can be said that visibly collapsed cakes had a higher residual moisture content than uncollapsed cakes. Lyophilisates without NaCl were always drier than lyophilisates with NaCl. As the after-drying time increased the residual moisture content decreased, and after 10 hrs. at 20° C. the residual moisture content was about 2%. Verum and placebo were comparable in their residual moisture content. At the start of the main drying the product temperature (40° C.) was below the Tg′ (−34° C.). Under the main drying conditions of −20° C. and 0.05 mbar optically suitable lyophilisates were obtained. The optimised lyophilisation programme is summarised in Table 18.

Lyophilisation of DC30 Lipoplexes:

The DC30 lipoplexes prepared according to Example 3 and packaged according to Example 5 were then lyophilised. The lyophiliser used was the Lyo Com 5018 made by Messrs Hof (Lohra, DE). The total duration of the lyophilisation process was 64 hrs. The vacuum was regulated using a vacuum valve. The vacuum measuring probe was a probe made by Messrs Pirani (Thyracont Elektronic GmbH, Passau, DE). Drying was done without freeze-drying sheets. The containers were sealed under a pressure of 800 mbar. The precise lyophilisation programme is detailed in Table 18.

TABLE 18

| | | Intended | |
|---|---|---|---|
| | Duration (hh:mm) | temperature (° C.) | vacuum (mbar) |
| loading | 00:00 | 5 | 1000 |
| freezing | 00:55 | −50 | 1000 |
| | 02:00 | −50 | 1000 |
| main drying | 00:05 | −50 | 0.05 |
| | 01:30 | −20 | 0.05 |
| | 47:00 | −20 | 0.05 |
| after-drying | 02:00 | 30 | 0.05 |
| | 10:00 | 30 | 0.05 |
| removing | 00:30 | 5 | 0.05 |
| total time | 64:00 | — | |

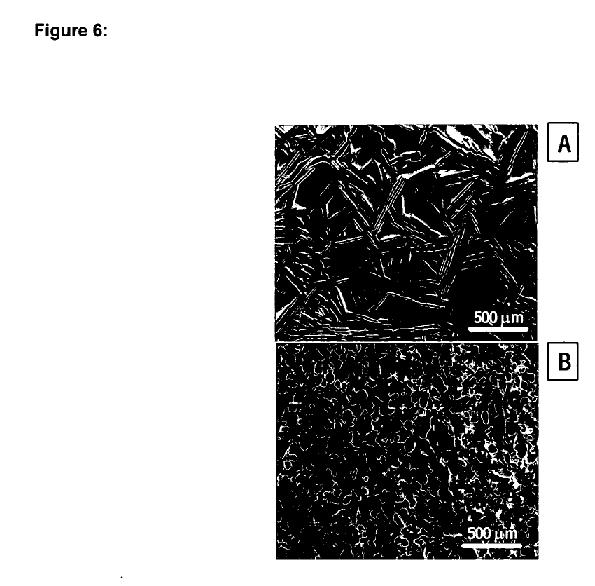

The end product (vial) was removed from the lyophiliser and the vial was flanged. It was stored at 2-8° C. The lyophilisation cake formed did not collapse. FIGS. 6A and 6B show SEM (Scanning Electron Microscope) photographs of a lyophilisation cake. FIG. 6A shows the surface morphology of the lyophilisation cake. FIG. 6B shows a detail of the lyophilisation cake itself. This type of lyophilisation cake has very short reconstitution times of a few seconds.

The residual moisture content (determined by Karl Fischer titration) of the DC30 lipoplexes in the batches from Table 9 are shown in Table 19. In each case 5 vials were selected per batch and their residual moisture content was determined.

TABLE 19

| | batch 1 (%) | batch 2 (%) | batch 3 (%) |
|---|---|---|---|
| sample | 1 extrusion | 2 extrusions | 3 extrusions |
| 1 | 0.77 | 0.76 | 0.74 |
| 2 | 0.95 | 0.70 | 0.66 |
| 3 | 0.74 | 0.75 | 0.83 |
| 4 | 0.73 | 1.05 | 0.52 |
| 5 | 0.76 | 0.96 | 0.73 |
| average | 0.79 | 0.84 | 0.70 |
| SD | 009 | 0.15 | 0.12 |
| VC | 11.5 | 18.15 | 16.58 |

SD: standard deviation,
VC: variation coefficient

The residual moisture content of the lyophilisates was ≦3%. No significant difference in the residual moisture content between the individual batches can be seen. It is known that the residual moisture content has a considerable influence on the stability of the product.

### Example 8

### Lyophilisation of Homogeneous Lipoplexes Consisting of DOPE/DAC-Chol 70/30 (DAC30) and Nucleic Acid

The lyophilisation was carried out in a Lyo Epsilon 2-12D lyophiliser, Messrs. Christ (Osterode, DE), in the Example described. The vacuum was regulated using a Pirani probe. The lyophilisation was done without sheets. Precise details of the lyophilisation programme are contained in Table 20.

TABLE 20

| | time | temperature | pressure |
|---|---|---|---|
| Process step | (hh:mm) | (° C.) | (mbar) |
| loading | 00:00 | +5 | 1000 |
| freezing | 00:55 | −50 | 1000 |
| freezing | 02:00 | −50 | 1000 |
| main drying | 00:05 | −50 | 0.05 |
| main drying | 01:30 | −20 | 0.05 |
| main drying | 47:00 | −20 | 0.05 |
| after-drying | 2:00 | +30 | 0.05 |
| after-drying | 10:00 | +30 | 0.05 |
| removal | 00:30 | +5 | 0.05 |
| total time | 64:00 | — | — |

The primary packaging was as in Example 5. The residual moisture content data (determined according to Karl Fischer) of DAC30 lipoplexes are detailed in Table 21 (for n=10 vials). The water content of lipoplexes is an important factor for the long-term stability of the product.

TABLE 21

| sample | water content in % |
|---|---|
| 1 | 0.65 |
| 2 | 0.54 |
| 3 | 0.71 |
| 4 | 0.65 |
| 5 | 0.78 |
| 6 | 0.65 |
| 7 | 0.71 |
| 8 | 1.29 |
| 9 | 0.85 |
| 10 | 0.90 |
| average | 0.77 |

The result of the residual moisture content for the DAC30 lipoplexes was again less than 1%. After reconstruction of the lyophilisates with 1.5 ml WFI the diameter of the DAC30 lipoplexes was determined. Table 22 shows the results for 4 independent batches using the method of preparation described above. Both the data of the lipoplex size 30 min after production (before Lyo) and after lyophilisation (after Lyo) are shown (cf. also Table 10).

TABLE 22

| | |
|---|---|
| Before Lyophilisation | After Lyophilisation |

| batch | size (nm) | Poly-Index | size (nm) | Poly-Index |
|---|---|---|---|---|
| 1 | 309 | 0.27 | 313 | 0.29 |
| 2 | 300 | 0.28 | 303 | 0.32 |
| 3 | 312 | 0.24 | 310 | 0.25 |
| 4 | 320 | 0.23 | 320 | 0.26 |

The lyophilisation programme used does not have a destabilising effect on the lipoplex size.

As the entire process was (may be) carried out under aseptic conditions, a pathogen count was done on the DAC30 lipoplex end product. The pathogen count was determined using the method in the European and US Pharmacopoeias. Table 23 shows the results for different batches of DAC30 lipoplexes.

TABLE 23

| lipoplexes after | pathogen count (cfu) | pathogen count (cfu) |
|---|---|---|
| Lyo | per mL/g | per mL/g |
| batch | bacteria | fungi |
| batch 1 | <1/2.5 Vials | <1/2.5 Vials |
| batch 2 | <1/2.5 Vials | <1/2.5 Vials |
| batch 3 | <1/2.5 Vials | <1/2.5 Vials |
| batch 4 | <1/2.5 Vials | <1/2.5 Vials |

cfu: colony forming unit

### EXAMPLE 9

### Bioactivity and In Vitro Transfection of Lipoplexes

Influence of the Sequence of Mixing the Nucleic Acid Solution and Liposome Dispersion on the Product Quality:

The order in which the nucleic acid solution and the liposome dispersion are mixed (lipid to nucleic acid=LtoD, nucleic acid to lipid=DtoL) (L: lipid, D: DNA) affects the stability of the lipoplexes and also their transfection properties. This depends partly on the lipid used, on the ratio of lipid to nucleic acid, total lipid concentration and formulation buffer.

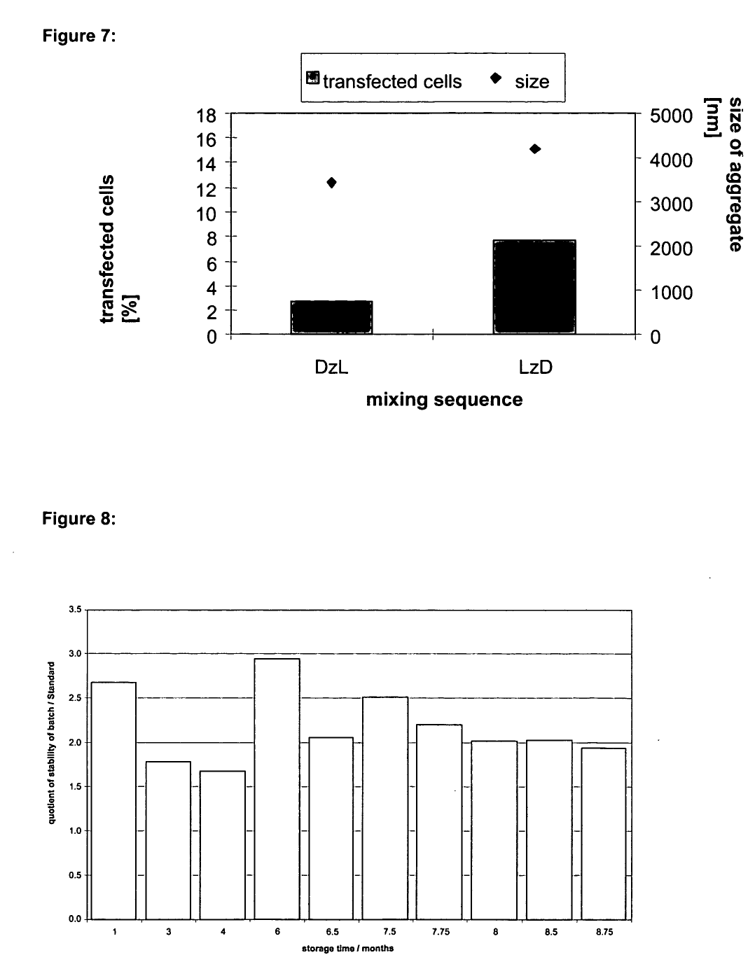

FIG. 7 shows the results by means of the example of the commercially obtainable lipid Lipofectin (Invitrogen life technologies, Carlsbad, USA). As FIG. 7 shows, both the size of the lipoplex and the transfection efficiency varied (determined by the expression of EGFP). The latter is very strongly influenced by the manner and method of combining and mixing the two solutions. Lipoplexes prepared by LtoD had a transfection efficiency which was double that of the DtoL lipoplexes. The particle size also varied (FIG. 7) and depended on the mixing process.

Similar tests were also carried out with the lipid DAC30, and to summarise it can be said that the DtoL lipoplexes transfect rather better than LtoD lipoplexes. In the case of Lipofectin the opposite mixing sequence gave better results. Both methods of preparation (DtoL and LtoD) had a tendency to cause aggregation and clouding of the lipoplex solution. The lipoplexes thus prepared were unstable in a liquid formulation and are thus unsuitable for further processing (decanting and subsequent lyophilisation). Moreover, these two methods (DtoL and LtoD) yielded lipoplexes with non-reproducible particle sizes. The particle sizes varied from batch to batch by more than 300%.

Pre-Treatment of the Liposomes with Respect to the Lipoplex Transfection Qualities:

The pre-treatment of the liposomes, extruded compared with non-extruded, has an effect on the transfection efficiency and particle size of the lipoplexes produced. Table 24 shows the transfection efficiency (expressed as % of transfected cells) of DAC30/pAH7-EGFP 4:1 (w/w) lipoplexes, which was carried out by complexing the plasmid with liposomes which were either non-extruded or extruded 1× through an 800 nm extrusion membrane and mixed using the Y-shaped member. The transfection efficiency for lipoplexes prepared with extruded liposomes was twice that of lipoplexes prepared from unextruded liposomes.

TABLE 24

transfection efficiency/%

| | unextruded | extruded |
|---|---|---|
| HA SMC | 3.7 | 7.9 |
| A-10 SMC | 12.0 | 26.4 |

HA SMC = human aorta smooth muscle cell,
A-10 SMC = rat smooth muscle cell

Bioactivity of the Lipoplex Batches Prepared According to Example 4:

The bioactivity (transfection property) of lipoplexes consisting of DAC30 and pMCP-1 plasmid in a mass ratio of 5:1 was tested by transfection of BHK21 cells. The expressed MCP-1 protein of transfected cells was measured using a BD OptEIA™ Human MCDA ELISA Kit, BD Biosciences Pharmingen. The total protein was determined using a commercially obtainable test (BCA, bicinchoninic acid).

$$Potency = \frac{conc . pMCP - 1 in pg \text{/} mL}{conc . protein in μg \text{/} mL}$$

Table 25 shows that the bioactivity of the freshly prepared lipoplexes (before Lyo) and of the lyophilised lipoplexes (after Lyo) was comparable. The ratio of the potency before/after lyophilisation was ˜1.10 and thus indicates that the bioactivity is unaffected.

TABLE 25

| | 6 well plate | |
|---|---|---|
| | lipoplex bioactivity | |
| | before lyophilisation | after lyophilisation |
| | Potency | Potency |
| batch | MW (pg MCP-1/μg protein) | MW (pg MCP-1/μg protein) |
| undiluted | 1182 | 1072 |
| diluted 1:2 | 819 | 746 |
| diluted 1:5 | 312 | 314 |

The ratio of expressed protein before and after lyophilisation for four independent batches is detailed in Table 26. Once again it is clear that the bioactivity of the lipoplexes is maintained.

TABLE 26

| | Quotient of protein expression |
|---|---|
| batch | before/after lyophilisation |
| 1 | 1.11 |
| 2 | 1.02 |
| 3 | 1.02 |
| 4 | 1.12 |

### Example 10

### Long-Term Stability of DAC30 μLipoplexes

The storage stability of the lipoplexes DAC30/pMCP-1 5:1 (w/w) (as lyophilisate) was determined at various temperatures (see FIG. 8). The following parameters were determined: the size of the lipoplexes, homogeneity (polydispersity index PI), DNA content, DNA integrity of the ccc form, residual moisture content (given as a quotient: measured value/zero value) and bioactivity (expressed as the transfection efficiency of the test batch, based on an internal standard). Storing these lipoplexes at 2-8° C. for 8 months (Table 27) shows that the size of the lipoplexes is in the range from 300-330 nm, with PI<0.3. The DNA content and also the integrity of the DNA (expressed as the % ccc form) scarcely altered within the scope of the accuracy of measurement. The residual moisture content did not change either.

TABLE 27

| | | | | | DNA | |
|---|---|---|---|---|---|---|
| | | | | DNA | integrity | residual |
| | sampling | size | Poly- | content | ccc form | moisture |
| T (° C.) | time | (nm) | Index | (μg/ml) | (%) | ratios |
| 4° C. | 0 months | 310 | 0.25 | 26 | 74 | 1 |
| | 4 months | 310 | 0.26 | 29 | 77 | 0.9 |
| | 8 months | 319 | 0.26 | 27 | 77 | 1.06 |
| 25° C. | 4 months | 328 | 0.25 | 25 | 77 | 1.1 |

T = storage temperature, nominal DMA content = 25 μg/ml, residual moisture
ratios = measured value/zero value

FIG. 8 shows the results for the bioactivity of the lipoplexes (stored at 4° C.) over a period of 8 months. It will be seen that the quotient of the bioactivity of the batch to be tested based on an internal standard includes values of around 2. The storage of the liquid lipoplexes at 37° C. leads to a drastic reduction in bioactivity after only about 2 months. The quotient is only 0.1.

The storage of the lipoplexes in lyophilised form at 37° C. shows that the quotient was 0.82 after 1 month and 0.5 after 2.5 months. Storage of the lipoplexes (lyophilisate) at 25° C. still exhibits a bioactivity quotient of more than 2 after 4 months.

1. A liposome mixture having a polydispersity index value of ≦0.60, wherein said mixture comprises liposomes with a defined size of between 250 and 800 nm and contain a cationic lipid and a neutral amphiphile.

2. The liposome mixture according to claim 1, wherein the cationic lipid is DC-Chol ((3-beta[N(N′,N′-dimethylaminoethane) carbamoyl]cholesterol)) o r DAC-Chol ((3-beta[N(N,N′-dimethylamino-ethane)carbamoyl]cholesterol).

3. The liposome mixture according to claim 1, wherein the neutral amphiphile is a choline derivative selected from dimyristoylphosphatidylcholine (DMPC), dipalmitoylphosphatidylcholine (DPPC) and dioleoylphosphatidylcholine (DOPC) or an ethanolamine derivative selected from dimyristoylphosphatidylethanolamine (DMPE), dipalmitoylphosphatidyl-ethanolamine (DPPE) and dioleoylphosphatidylethanolamine (DOPE).

4. The liposome mixture according to claim 1, wherein the liposomes comprise DOPE as neutral amphiphile and DC-Chol and DAC-Chol as cationic lipid with the mass ratio of DOPE to the cationic lipid being 70:30.

5. A process for preparing homogeneous liposomes, wherein a lipid suspension is extruded through a porous membrane in a continuous process under low pressure conditions of less than 3×10⁵ Pa.

6. The process according to claim 5, wherein the homogeneous liposomes contain a cationic lipid and a neutral amphiphile.

7. The process according to claim 6, wherein the cationic lipid is a cholesterol derivative.

8. The process according to claim 7, wherein the cholesterol derivative is DC-Chol ((3-beta[N(N′,N′-dimethylaminoethane) carbamoyl]cholesterol)) o r DAC-Chol ((3-beta[N(N,N′-dimethylamino-ethane)carbamoyl]cholesterol)).

9. The process according to claim 6, wherein the neutral amphiphile is a choline derivative selected from dimyristoylphosphatidylcholine (DMPC), dipalmitoylphosphatidylcholine (DPPC) and dioleoylphosphatidylcholine (DOPC) or an ethanolamine derivative selected from (dimyristoylphosphatidylethanol-amine (DMPE), dipalmitoylphosphatidylethanolamine (DPPE) and dioleoylphosphatidyl-ethanolamine (DOPE).

10. The process according to claim 6, wherein liposomes contain or consist of DOPE as neutral amphiphile and DC-Chol and/or DAC-Chol as cationic lipid with the mass ratio of DOPE to cationic lipid being 70:30.

11. The process according to claims 5, wherein the liposome concentration in the lipid suspension is 0.04-5 mg/ml.

12. The process according to claim 5, wherein lipid suspension is a suspension of a cationic lipid and a neutral amphiphile in an aqueous solution.

13. The process according to claim 12, wherein the suspension additionally contains salts, polymers and/or sugar compounds.

14. The process according to one claim 5, wherein the extrusion is carried out at a flow rate of between 10 and 250 ml/min.

15. The process according to claim 5, wherein the extrusion is carried out at ambient temperature.

16. The process according to claim 5, wherein the extrusion is carried out under aseptic conditions in a sealed system.

17. The process according to claim 5, wherein the porous membrane is a polycarbonate membrane.

18. The process according to claim 5, wherein the porous membrane has pores ranging in size from 600 nm to 900 nm.

19. The process according to claim 5, wherein the lipid suspension is extruded continuously between 2 and 20 times through the porous membrane.

20. A liposome obtainable by a process according to claim 18.

21. A liposome obtainable by a process according to claim 19.

22. A process for preparing lipoplexes wherein the liposomes according to claim 20 or 21 are mixed with nucleic acid molecules.

23. The process according to claim 22, wherein the mixing of liposomes and nucleic acid molecules is done through a Y-shaped member which allows equal volumes of the liposomes and nucleic acid molecules to be combined evenly and continuously.

24. The process according to claim 22, wherein concentration of liposomes during mixing is between 0.02 and 1 mg/ml.

25. The process according to claim 22, wherein liposomes-nucleic acid charge ratio (+/−) is between 4 and −0.01.

26. The process according to claim 25 wherein the liposomes-nucleic acid charge ratio (+/−) is between 0.75 and 1.25.

27. The process according to claim 22, wherein the liposomes and nucleic acid are mixed at a flow rate of 20 to 800 ml/min.

28. The process according to claim 27, wherein the liposomes and nucleic acid are mixed at a flow rate of 100 to 500 ml/min.

29. A process for preparing homogeneous lipoplex mixtures comprising lipoplexes measuring 250-600 nm and having a polydispersity index of ≦0.5 comprising the steps of:
(a) extruding a lipid suspension containing DC30 or preferably DAC30, in a continuous process through a 600-900 nm membrane, while the lipid concentration in the lipid suspension is between 0.04-5 mg/ml, wherein the lipid suspension is extruded through the membrane at least once; and
(b) mixing the liposome mixture thus prepared with nucleic acid molecules which have previously been filtered sterile, using a Y-shaped member at a continuous flow rate of 20-800 ml/min and a liposome-nucleic acid charge ratio (+/−) of 4-0.01.

30. The process according to claim 29 wherein the lipoplexes are prepared under aseptic conditions.

31. A lipoplex mixture comprising lipoplexes with a defined size of between 250 and 600 nm, said lipoplexes comprising a liposome mixture according to claim 1 and nucleic acid molecules, wherein the polydispersity index of the lipoplex mixture is ≦0.50.

32. The lipoplexes obtainable by a process according to claims 22.

33. The lipoplexes obtainable by a process according to claims 29.

34. A process for lyophilising lipoplexes according to claim 32 in the presence of a suitable stabiliser comprising the steps of
a) freezing the lipoplex mixture to a temperature of ≦−50° C.;
b) drying the lipoplex mixture at approximately −20° C. for at least 35 hours;
c) after-drying the lipoplex mixture at approximately 20° C. for at least 10 hours.

35. A process for lyophilising lipoplexes according to claim 33 in the presence of a suitable stabiliser comprising the steps of
d) freezing the lipoplex mixture to a temperature of ≦−50° C.;
e) drying the lipoplex mixture at approximately −20° C. for at least 35 hours;
f) after-drying the lipoplex mixture at approximately 20° C. for at least 10 hours.

36. A process for lyophilising a lipoplex mixture in the presence of a suitable stabiliser comprising the steps of
a) freezing the lipoplex mixture to a temperature of ≦−50° C. at a temperature lowering rate of approximately ≦1° C./min;
b) incubating the lipoplex mixture at ≦−50° C. for at least 2 hours;
c) heating the lipoplex mixture to approximately −20° C. at a heating rate of approximately ≦0.3° C./min;
d) drying the lipoplex mixture at approximately −20° C. for at least 35 hours;
e) heating the lipoplex mixture from about −20° C. to about 20° C. at a heating rate of approximately ≦0.44° C./min;
f) after-drying the lipoplex mixture at about 20° C. for at least 10 hours.

37. The process according to claim 34, wherein the drying is carried out at a pressure between 0.025 and 0.05 mbar.

38. The process according to claim 35, wherein the drying is carried out at a pressure between 0.025 and 0.05 mbar.

39. Lipoplex lyophilisates obtainable by a process according to claim 34.

40. Lipoplex lyophilisates obtainable by a process according to claim 35.

41. Lipoplex lyophilisates obtainable by a process according to claim 36.

42. A method of employing lipoplexes according to claim 32, lipoplex mixtures according to claim 31 or lipoplex lyophilisates according to any one of claim 39-42 in preparing a pharmaceutical compositions(s) in gene therapy.
